(19) United States
(12) Patent Application Publication
(10) Pub. No.: US 2015/0247843 A1
(43) Pub. Date: Sep. 3, 2015

(54) METHOD OF DETECTING TARGET SUBSTANCE

(71) Applicants: Kimiyasu TAKOH, Tokyo (JP); Minoru ASOGAWA, Tokyo (JP)

(72) Inventors: Kimiyasu TAKOH, Tokyo (JP); Minoru ASOGAWA, Tokyo (JP)

(73) Assignee: NEC CORPORATION, Tokyo (JP)

(21) Appl. No.: 14/713,634

(22) Filed: May 15, 2015

Related U.S. Application Data
(62) Division of application No. 13/257,197, filed on Oct. 12, 2011.

(30) Foreign Application Priority Data
Mar. 17, 2009 (JP) 2009-065319

(51) Int. Cl. G01N 33/543; C12N 15/10; G01N 33/53

(52) U.S. Cl. CPC G01N 33/54306; G01N 33/5308; C12N 15/1048; G01N 2458/30

(57) ABSTRACT

Provided are a method of detecting a target substance and the like using a probe with a simple design for target substance detection. The method of detecting a target substance of the present invention includes a step of providing a probe 4 in which an aptamer-bindable substance 1 and a labeling substance 2 are each bound to a linker 14 that is immobilizable to a support 5 and an aptamer 6 is bound to the aptamer-bindable substance 1; and a step of detecting separation of the aptamer 6 based on the labeling substance 2 by separating the aptamer 6 from the aptamer-bindable substance 1 through binding between a target substance 8 in a sample and the aptamer 6 wherein the probe 4 is immobilized to the support 5.

### CROSS-REFERENCE TO RELATED APPLICATIONS

This application is a Divisional of U.S. patent application Ser. No. 13/257,197, filed on Oct. 12, 2011, which is a National Stage of International Application No. PCT/JP2010/054543, filed on Mar. 17, 2010, which claims priority from Japanese Patent Application No. 2009-065319 filed Mar. 17, 2009, the contents of all of which are incorporated herein by reference in their entirety.

### TECHNICAL FIELD

The present invention relates to a method of detecting a target substance, a probe, a target substance detection apparatus, a method of screening an aptamer, and an aptamer screening apparatus.

### BACKGROUND ART

Aptamers are nucleic acids (DNA, RNA, PNA, and the like) or peptides that bind to specific substances, and receive attention in various fields including medicine, biotechnology, and the like. An aptamer can be obtained from a nucleic acid library by screening a sequence that shows a significant binding ability using the Systematic Evolution of Ligands by Exponential Enrichment (SELEX) method, for example. Utilizing this specific binding ability of the aptamer, sensors for disease diagnoses, environmental monitoring, checkups of belongings, and the like have been developed. Such sensors detect electrochemical changes, optical changes, mass changes, and the like due to binding between target substances and aptamers. Among these sensors, from the viewpoint of downsizing of the apparatuses, sensors for detecting electrochemical changes have been most commonly developed.

Sensors using aptamers are disclosed, for example, in the following Patent Documents 1 to 4 and the following Non-Patent Documents 1 to 4.

Patent Document 1 discloses a bioelectrical sensor. In this sensor, one end of an aptamer is modified with an electrode reactive substance and the other end is immobilized to an electrode.

Patent Document 2 discloses a method of detecting a target substance using a nucleic acid probe. In this method, a complex of a labeled target substance and a nucleic acid ligand is dissociated and detection is performed based on a labeling substance.

Patent Document 3 discloses a method of detecting macromolecular biopolymers using an electrode structure. In this method, a complementary strand modified with an electrode reactive substance is hybridized to an aptamer that is immobilized to an electrode.

Patent Document 4 discloses a method of detecting a nucleic acid and/or a polypeptide. In this method, a target nucleic acid or a polypeptide is labeled with a ligand complex.

Non-Patent Document 1 discloses an electrochemical detection method of an aptamer biosensor. In this method, an intercalator having electrode reactivity is being inserted in an aptamer that is immobilized to an electrode surface.

Non-Patent Document 2 discloses a target reactive electrochemical aptamer switch. In this sensor, one end of an aptamer is modified with an electrode reactive substance and the other end is immobilized to an electrode, and a complementary strand is hybridized to the aptamer.

Non-Patent Document 3 discloses an aptamer electrochemical sensor. In this sensor, a complementary strand modified with an electrode reactive substance is hybridized to an aptamer that is immobilized to an electrode.

Non-Patent Document 4 discloses an aptamer electrochemical sensor using a DNA oligonucleotide complementary to an aptamer. In this sensor, an aptamer is hybridized to a complementary strand, one end of which is modified with an electrode reactive substance and the other end of which is immobilized to an electrode.

### RELATED ART DOCUMENT

### Patent Document

Patent Document 1: US 20070020641 A
Patent Document 2: JP 2006-129866 A
Patent Document 3: JP 2004-524534 A
Patent Document 4: JP 2007-534961 A

### Non-Patent Document

Non-Patent Document 1: Gyeong Sook Bang, et al., Biosens. Bioelectronics, vol. 21 (2005) p. 863
Non-Patent Document 2: Xiaolei Zuo, et al., JACS, vol. 129 (2007) p. 1042
Non-Patent Document 3: Yi Xiao, et al., JACS, vol. 127 (2005) p. 17990
Non-Patent Document 4: Ying Lu, et al., Anal. Chem., vol. 80 (2008) p. 1883

### SUMMARY OF INVENTION

### Problem to be Solved by the Invention

In the sensors described in these Patent Documents and Non-Patent Documents, aptamers or their complementary strands are used as probes. Therefore, there is a need to modify the aptamers and the like with electrode substances or functional groups for a crosslinking reaction with an electrode surface or to optimize the structure of the aptamers so as to be suitable for sensors. Accordingly, with respect to these sensors, the design, synthesis, and the like of the probe are complicated, and this results in an increase in cost.

Hence, the present invention is intended to provide a method of detecting a target substance and the like using a probe with a simple design. The present invention is also intended to provide a probe used for the method of detecting a target substance and the like. Further, the present invention is intended to provide a method of screening an aptamer and the like capable of easily obtaining an aptamer used for the method of detecting a target substance and the like.

### Means for Solving Problem

The method of detecting a target substance of the present invention includes:

a step of providing a probe comprising an aptamer-bindable substance, a labeling substance, and a linker immobilizable to a support wherein the aptamer-bindable substance and the labeling substance are each bound to the linker and an aptamer is specifically bound to the aptamer-bindable substance; and
a step of detecting the target substance by separating the aptamer from the aptamer-bindable substance through binding between a target substance in a sample and the aptamer and detecting separation of the aptamer based on the labeling substance wherein the probe is immobilized to a support via the linker.

The probe of the present invention used for the method of detecting a target substance according to the present invention includes:

an aptamer-bindable substance,
a labeling substance, and
a linker immobilizable to a support, wherein
the aptamer-bindable substance and the labeling substance are each bound to the linker,
the aptamer-bindable substance and the labeling substance are immobilizable to the support via the linker, and
an aptamer is specifically bindable to the aptamer-bindable substance.

The target substance detection apparatus of the present invention used for the method of detecting a target substance according to the present invention includes:

the probe according to the present invention;
a separation detecting unit for detecting separation of the aptamer from the aptamer-bindable substance due to binding between a target substance in a sample and the aptamer based on the labeling substance, and
a support that immobilizes the probe via the linker at the time of detecting the separation.

The method of screening an aptamer of the present invention includes:

a first step of supplying an aptamer candidate substance to the probe according to the present invention;
a second step of separating the aptamer candidate substance from the probe by immobilizing the probe to a support via the linker, supplying a target substance to the probe, and binding the aptamer candidate substance that is bound to the probe to the target substance; and
a third step of recovering the aptamer candidate substance separated.

The aptamer screening apparatus of the present invention used for the method of screening an aptamer according to the present invention includes:

the probe according to the present invention;
a recovering unit for recovering an aptamer candidate substance separated from the probe due to binding between a target substance and the aptamer; and
a support that immobilizes the probe.

### Effects of the Invention

The present invention can provide a method of detecting a target substance wherein the design of a probe for detecting a target substance is simple. Further, the present invention can provide a probe capable of achieving the method of detecting a target substance and a target substance detection apparatus including the probe. Furthermore, the present invention can provide a method of screening an aptamer and the like capable of easily obtaining an aptamer that can be used for the method of detecting a target substance and the like.

### BRIEF DESCRIPTION OF DRAWINGS

FIGS. 1A to 1G are views illustrating a mechanism of an embodiment of the method and apparatus of the present invention.

FIGS. 2A to 2G are views illustrating a mechanism of another embodiment of the method and apparatus of the present invention.

FIGS. 3A to 3G are views illustrating a mechanism of yet another embodiment of the method and apparatus of the present invention.

FIGS. 4A to 4G are views illustrating a mechanism of still another embodiment of the method and apparatus of the present invention.

FIGS. 5A to 5C are views illustrating a mechanism of a further embodiment of the method and apparatus of the present invention.

FIGS. 6A to 6F are views illustrating a mechanism of yet further embodiment of the method and apparatus of the present invention.

FIGS. 7A to 7E are views illustrating a mechanism of still further embodiment of the method and apparatus of the present invention.

### DESCRIPTION OF EMBODIMENTS

### [Method of Detecting Target Substance]

The method of detecting a target substance of the present invention includes the step of providing a probe (hereinafter, this may also be referred to as the “probe provision step (A)”) and the step of detecting (hereinafter, this may also be referred to as the “detection step (B)”), and thereby can detect a target substance as follows, for example.

First, a probe to which an aptamer is specifically bound is provided in the probe provision step (A). The probe includes an aptamer-bindable substance, a labeling substance, and a linker immobilizable to a support. The aptamer-bindable substance and the labeling substance are each bound to the linker, and an aptamer is specifically bindable to the aptamer-bindable substance. The probe in which the aptamer is specifically bound to the aptamer-bindable substance can be obtained by performing an aptamer binding step (A-0) of binding the aptamer to the probe in which an aptamer has not been bound to the aptamer-bindable substance yet prior to the probe provision step (A), for example. Also, the probe to which the aptamer is preliminarily bound may be purchased prior to the probe provision step (A). In the detection method of the present invention, following that, separation of the aptamer due to binding between a target substance in a sample and the aptamer is detected in the detection step (B). In other words, in the detection method of the present invention, for example, a sample is supplied (added) to the probe to which the aptamer is bound. Thereby, in the case where the sample contains a target substance to which the aptamer is bindable, the target substance approaches to the aptamer that is bound to the aptamer-bindable substance in the probe. Then, the aptamer binds to the target substance and is separated from the aptamer-bindable substance. In this manner, in the detection method of the present invention, by binding the aptamer to the aptamer-bindable substance in the probe and then detecting separation of the aptamer from the aptamer-bindable substance due to binding between the target substance and the aptamer, the target substance is detected. Therefore, there is no need to modify the aptamer with a functional group or a labeling substance for detecting the target substance, for example, and thereby the design of the probe for detecting a target substance is simplified. Further, since the detection method of the present invention can detect the target substance without depending on a conformational change of the aptamer, for example, the method can be applied to various aptamers and target substances and has high general versatility.

The detection mechanism of a target substance according to the detection method of the present invention can be described as follows, for example. That is, according to the detection method of the present invention, in the detection step (B), the dynamic behavior of the probe in a reaction phase changes between the case where the aptamer is not bound to the aptamer-bindable substance of the probe and the case where the aptamer is bound to the aptamer-bindable substance of the probe. Here, in the detection method of the present invention, at the time of detecting a target substance, since the probe including the target substance is immobilized on a support, the change of the dynamic behavior of the probe can be detected easily based on the labeling substance, and this makes it possible to detect the target substance easily. In the detection method of the present invention, for example, any substance that can show the change of the dynamic behavior of the probe at its external part can be used as the labeling substance. Examples of the labeling substance include signal producing substances that produce signals such as optical signals, electrical signals, and color signals. In this case, the change of the dynamic behavior of the probe can be detected by detecting the change of the signal that is produced by the signal producing substance before and after separation of the aptamer from the aptamer-bindable substance, for example. The change of the signal can be detected with any separation detecting unit that is capable of detecting the change of signal.

In the detection method of the present invention, for example, an electrode can be used as the support. In this case, for example, the target substance can be detected by immobilizing the probe provided in the probe provision step (A) to an electrode and detecting separation of the aptamer in the detection step (B) as an electrical reaction between the labeling substance and the electrode. In other words, in this case, the change of the dynamic behavior of the probe in a reaction phase between the case in which the aptamer is separated from the probe and the case in which the aptamer is kept bound to the probe can be detected as an electrical reaction using the electrode.

Further, in the detection method of the present invention, for example, an electrode reactive substance can be used as the labeling substance. In this case, for example, by comparing the detection value of the electron transfer between the electrode reactive substance and the electrode prior to supply of the target substance to the probe and the detection value of the electron transfer between the electrode reactive substance and the electrode after supply of the target substance to the probe, separation of the aptame in the detection step is detected, and thereby the target substance can be detected. The detection value of the electron transfer shows, for example, the efficiency of the electron transfer between the electrode reactive substance and the electrode. The electron transfer can be detected with any electron transfer detecting unit that is capable of detecting the electron transfer between the electrode reactive substance and the electrode, for example. According to this embodiment, since the probe includes the electrode reactive substance, for example, there is no need to modify the aptamer with the electrode reactive substance. Further, since the aptamer can be immobilized to the electrode by the probe, for example, there is no need to modify the aptamer with a functional group or the like for formation of a crosslinking reaction with an electrode surface. Accordingly, the target substance can be detected very simply. Further, this embodiment shows a signal-increasing type detection reaction in which the detection value of the electron transfer increases as the target substance increases, for example, and can achieve a high signal/noise ratio (S/N ratio). The detection mechanism of the target substance in this embodiment can be described as follows, for example. However, the following descriptions are mere illustrations and do not limit the present invention. In the detection step (B), when the target substance binds to the aptamer and the aptamer is separated from the aptamer-bindable substance, the mobility of the probe in a reaction phase increases. On the other hand, for example, in the case where the target substance is not a target substance to which the aptamer is bindable, since the aptamer bound to the aptamer-bindable substance does not bind to the target substance, the aptamer is not separated from the aptamer-bindable substance. Therefore, the mobility of the probe in the reaction phase remains low. Here, in this embodiment, the probe is immobilized to the electrode via the linker. Therefore, in the case where the mobility of the probe is high, the contact frequency between the electrode reactive substance of the probe and the electrode is high. On the other hand, in the case where the mobility of the probe is low, the contact frequency between the electrode reactive substance of the probe and the electrode is low. Here, the higher the contact frequency between the electrode reactive substance and the electrode is, the higher the detection value of the electron transfer between the electrode reactive substance and the electrode. In other words, the detection value of the electron transfer in the case where the aptamer is not bound to the aptamer-bindable substance is higher than that in the case where the aptamer is bound to the aptamer-bindable substance. Thus, the target substance can be detected by detecting the change of the electron transfer.

The detection method of the present invention may further include a probe immobilization step of immobilizing the probe to the support prior to the probe provision step (A), for example. Also, the probe immobilized to the support may be purchased. In the case where the detection method of the present invention includes the aptamer binding step (A-0), the probe immobilization step can be performed, for example, before the aptamer binding step (A-0), during the step (A-0), or after the step (A-0). Thereby, the procedure of the detection method of the present invention can be simplified if necessary.

In the detection method of the present invention, for example, a known detection value may be used as the detection value of the electron transfer between the electrode reactive substance and the electrode prior to supply of the target substance to the probe, and the known detection value may be compared with the detection value of the electron transfer between the electrode reactive substance and the electrode after supply of the target substance to the probe. Thereby, for example, detection of the electron transfer between the electrode reactive substance and the electrode prior to supply of the target substance becomes unnecessary. The known detection value can be calculated, for example, from the density of the probe on the electrode in the probe provision step (A) and the ratio between the number of probes bound to aptamers and the number of probes not bound to aptamers. That is, thereby, the detection value of the electron transfer between the electrode reactive substance and the electrode can be obtained preliminarily using an electrode to which the probe and the aptamer are bound under known conditions. Then, the target substance can be detected from the difference between the known detection value and the detection value of the electron transfer detected in the detection step (B).

The detection method of the present invention may further include a step of removing an aptamer that is not bound to the aptamer-bindable substance prior to the detection step (B), for example. Thereby, the target substance can be prevented from binding to an aptamer that is not bound to the aptamer-bindable substance. As a result, the reproducibility of the amount of the aptamer separated from the aptamer-bindable substance can be improved and a detection result that reflects the presence of the target substance more correctly can be obtained.

The detection method of the present invention can be performed, for example, in a liquid such as a solution or the like. The liquid is not particularly limited as long as binding between the aptamer and the aptamer-bindable substance and separation of the aptamer from the aptamer-bindable substance occur therein, for example. In other words, the conditions such as the composition of the liquid in which the detection method of the present invention is performed and a temperature, a pH, and an electrolyte in each of the steps of the detection method of the present invention are not limited as long as the binding and separation can occur. As the conditions, for example, conditions commonly used for the SELEX method can be used, and the conditions can be set suitably such that the binding and separation occur. In the case where the detection step (B) is performed by detecting the electron transfer, the electrolyte concentration of the liquid is preferably not too low so that the detection accuracy of the electron transfer in the detection step (B) is not impaired.

[Probe]

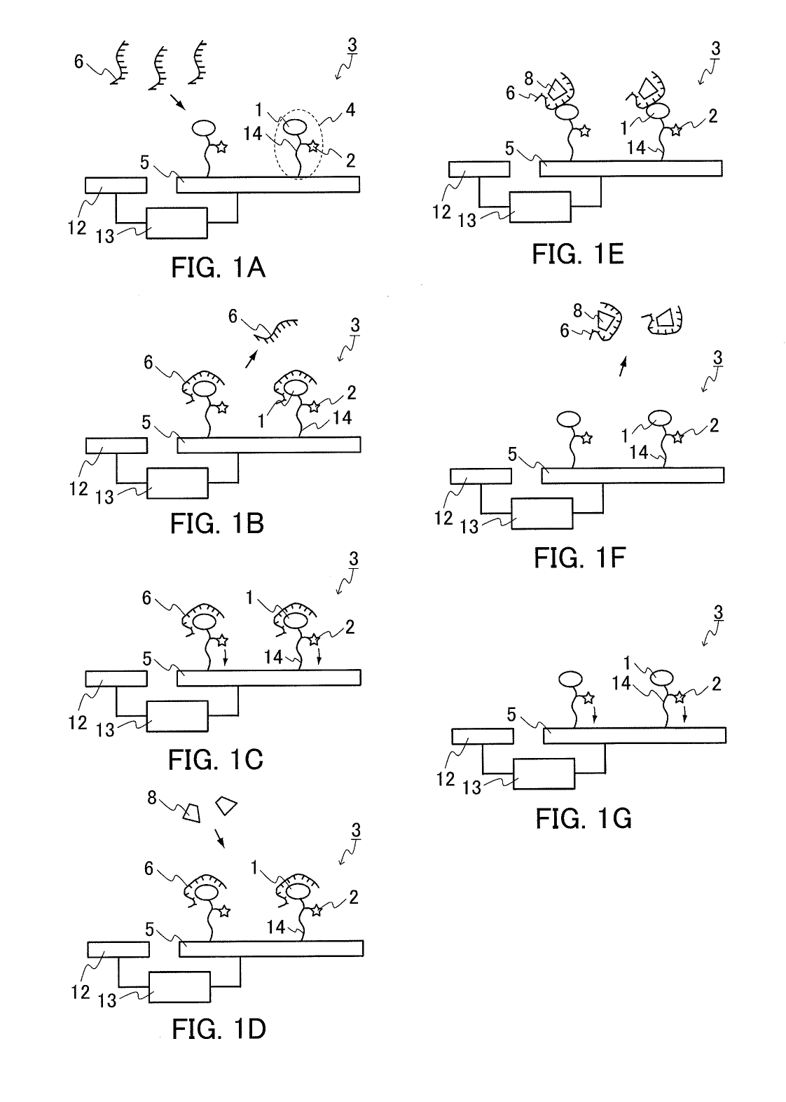

The probe is not particularly limited as long as it includes an aptamer-bindable substance, a labeling substance, and a linker immobilizable to a support, the aptamer-bindable substance and the labeling substance are each bound to the linker, and an aptamer is specifically bindable to the aptamer-bindable substance. In the detection method of the present invention, the probe is immobilized to a support at the detection step as described above. The probe may be immobilized to an electrode, for example. Thereby, for example, binding of the aptamer to the probe and separation of the aptamer from the probe can be detected as an electrical reaction between the labeling substance and the electrode using the electrode. In the probe, the aptamer-bindable substance and the labeling substance each may be bound directly to the linker or one of them may be bound directly to the linker and also bound to the other, for example. The probe preferably has a functional group that can be immobilized to an electrode surface at at least one end of the linker, for example. FIG. 1A shows the configuration of an example of the probe of the present invention. As shown in FIG. 1A, a probe 4 includes an aptamer-bindable substance 1 that is specifically bound to an aptamer 6 and a labeling substance 2, and the aptamer-bindable substance 1 and the labeling substance 2 are each bound to a linker 14 that is immobilizable to a support 5. In detection of a target substance, for example, the probe is applicable as long as it is immobilized to the support at the probe provision step (A). The molecular structure of the probe is not particularly limited, and can be designed suitably according to the size of the target substance, the size of the aptamer, and the property of the aptamer such as the chain length or the like. Further, for example, the probe may include a substance other than the aptamer-bindable substance, the labeling substance, and the linker as long as the effect of the present invention is not impaired. In the detection method of the present invention, in the case where an electrode reactive substance is used as the labeling substance and the probe is immobilized to an electrode, the probe moves in the vicinity of the electrode surface by the structural changes such as flexure and refraction due to diffusion, a thermal motion, and the like in a solution, for example. In accordance with the movement, the electrode reactive substance contained in the probe is brought into contact with the electrode surface and is separated from the electrode surface. That is, the higher the mobility of the probe is, the higher the contact frequency between the electrode reactive activity and the electrode surface. As a result, the detection value of the electron transfer between the electrode reactive substance and the electrode is increased. In contrast, the lower the mobility of the probe is, the lower the contact frequency between the electrode reactive substance and the electrode surface. As a result, the detection value of the electron transfer between the electrode reactive substance and the electrode decreases. Therefore, a signal-amplifying type detection embodiment in which the detection signal is amplified due to the presence of the target substance can be achieved by utilizing the foregoing dynamic behavior of the probe, for example. Further, a high S/N ratio can be achieved.

The aptamer-bindable substance is not particularly limited and can be any substance as long as it is capable of binding to the aptamer. For example, the aptamer-bindable substance is preferably the one having the structure that is identical to or similar to the whole or a part of the target substance. Further, from the viewpoint of general versatility, for example, in the case where the aptamer is a nucleic acid, preferably, the aptamer-bindable substance does not immobilize the nucleic acid of the aptamer by a nucleic acid having a base sequence complementary to the nucleic acid. However, even the nucleic acid having a base sequence complementary to the nucleic acid can be used as the aptamer-bindable substance in the detection method of the present invention. In the detection method of the present invention, as described above, the probe includes the linker. Therefore, for example, in the case where the aptamer is a nucleic acid, it is unnecessary for the aptamer and the complementary strand of the aptamer to have a hairpin structure or the like as described in the Non-Patent Document 4, and the design of the probe is simple. In the detection method of the present invention, the aptamer-bindable substance has an epitope that is identical to or similar to the whole or a part of the target substance, for example. The epitope is, for example, a substance that is specifically bindable to the aptamer. In the present invention, for example, the “epitope” refers to a part of a target substance to which the aptamer is specifically bindable. For example, an epitope that is similar to the epitope refers to a substance having the structure that is similar to the epitope to which an aptamer that is bindable to the target substance can be specifically bound. Examples of such an epitope that is similar to the epitope include the one that is obtained by removing a part of the epitope, the one that is obtained by substituting a part of the epitope with another functional group, and the one obtained by adding a new functional group to the epitope. In the present invention, in the case where a sample does not contain the target substance, the aptamer is immobilized to the probe by the epitope or an epitope that is similar to the epitope, and thereby the aptamer is not separated from the aptamer-bindable substance. In other words, the detection method of the present invention shows a high degree of selectivity to the target substance, for example. In the present invention, by adding the target substance to the aptamer-bindable substance to which the aptamer is bound, the aptamer binds to the target substance competitively and is separated from the probe. In the detection method of the present invention, for example, increasing the level of similarity between the structure of the aptamer-bindable substance and the structure of the epitope makes separation of the aptamer from the aptamer-bindable substance difficult. Thereby, the selectivity of the target substance can further be increased. On the other hand, for example, decreasing the level of similarity between the structure of the aptamer-bindable substance and the structure of the epitope makes separation of the aptamer from the aptamer-bindable substance easy. Thereby, for example, the amount of the aptamer separated is increased and the detection signal can further be increased. The level of similarity between the structure of the aptamer-bindable substance and the structure of the epitope can be designed appropriately depending on needed selectivity or detection sensitivity, for example. In the case where the aptamer has, for example, a double-stranded nucleic acid part, the aptamer-bindable substance is preferably a substance that specifically binds to the double-stranded nucleic acid part of the aptamer (In the present invention, this may also be referred to as a double-stranded nucleic acid aptamer-bindable substance). In the case where the aptamer-bindable substance is the double-stranded nucleic acid aptamer-bindable substance, for example, the target substance can be detected specifically without immobilizing a substance having an epitope that is identical to or similar to the whole or a part of a target substance to an electrode. Therefore, especially, in the case where the double-stranded aptamer-bindable substance is used as the aptamer-bindable substance, for example, the detection method of the present invention can be applied, to various aptamers that contain double-stranded sequence parts, and therefore general versatility can be increased. In the detection method of the present invention, preferably, such a double-stranded nucleic acid aptamer-bindable substance does not bind to a single stranded part of a nucleic acid, for example. Examples of the foregoing double-stranded nucleic acid aptamer-bindable substance include, but not limited to, intercalators and nucleic acid binding proteins. Examples of the intercalators include nitrogen-containing condensed cyclic compounds such as acridine and ethidium bromide; methylene blue; benzopyrene; actinomycin; nogalamycin; distamycin A; methidium; and derivatives thereof. Examples of the nucleic acid binding proteins include groove binder, zinc finger, and leucine zipper. The probe may contain one or more than one aptamer-bindable substance per molecule of the probe, for example. In the case where the probe includes more than one aptamer-bindable substance per molecule, for example, when the aptamer has a double-stranded nucleic acid part, more than one aptamer-bindable substance tends to bind to the double-stranded nucleic acid part. When more than one aptamer-bindable substance binds in this manner, for example, the binding strength between the aptamer and the probe is increased. By the use of such a probe, for example, a substance having a low binding strength with the aptamer is not detected and only a target substance having a higher binding strength is detected. Thereby, the selectivity of the detection method of the present invention is increased. However, if the number of aptamer-bindable substances per molecule of the probe is too large, binding between the target substance and the aptamer may be hindered and the selectivity may be decreased. Therefore, preferably, the number of aptamer-bindable substances is decided so that it does not exceed the binding strength between the target substance and the aptamer, for example. The number can be obtained by experiments, for example. Further, a substance having electrode reactivity may be used as the aptamer-bindable substance, for example. In this case, for example, since the aptamer-bindable substance also serves as the electrode reactive substance contained in the probe, the structure of the probe can be simplified and the production of a target substance detection apparatus can be simplified. Examples of the aptamer-bindable substance having electrode reactivity include, but not limited to, intercalators having electrode reactivity. Examples of the intercalators having electrode reactivity include methylene blue, quinone, acridine, and derivatives thereof.

As described above, the labeling substance is not limited as long as it can show the change of the dynamic behavior of the probe in a reaction system at its external part. Examples of the labeling substance include signal producing substances that produce signals such as optical signals, electrical signals, and color signals. An electrode reactive substance can be used as the labeling substance, for example. The electrode reactive substance is not limited as long as it can react with the electrode. The electrode reactive substance is preferably the substance having the following property: the detection value of the electron transfer between the substance and the electrode increases as the contact frequency with the electrode increases, for example. Examples of the foregoing electrode reactive substance include substances having oxidation-reduction potentials and catalysts. Examples of the catalysts include enzymes. In the case where the substance having an oxidation-reduction potential is used as the electrode reactive substance, for example, an electron is transferred therebetween through generation of an electrochemical reaction by the contact between the electrode and the electrode reactive substance. In the detection method of the present invention, for example, this electron transfer is detected for detecting a target substance. Specifically, in the case where the contact frequency between the electrode reactive substance and the electrode is increased, for example, an increase in the detection value of the electron transfer can be detected. The substance having an oxidation-reduction potential is preferably the one whose standard electrode potential according to a standard hydrogen electrode is between −0.6 V to +1.4 V, for example. In the case where the standard electrode potential is in the aforementioned range, for example, a reaction between the electrode and a substance other than the electrode reactive substance such as a solvent of a solution for the detection method of the present invention and dissolved oxygen can be suppressed. As a result, since a base line current is decreased, the change of the electron transfer between the electrode reactive substance and the electrode can be detected sensitively, and the detection sensitivity is increased. The electrode reactive substance is not particularly limited and examples thereof include metals such as Os, Fe, Ru, Co, Cu, Ni, Ti, V, Mo, Cr, Mn, Ag, Pd, and W; salts of the metals; complexes having ions of the metals as central metals; quinones such as hydroquinone and anthraquinone and derivatives thereof; methylene blue and derivatives thereof; pyrroles; and heterocyclic compounds such as pyridine and viologen. In the case where a catalyst is used as the electrode reactive substance, for example, the catalyst that has transferred electrons with a reactive substance is brought into contact with the electrode to cause an electrochemical reaction, and this results in the electron transfer between the electrode and the electrode reactive substance. Thereby, for example, in the case where the contact frequency between the electrode reactive substance and the electrode is increased, for example, an increase in the detection value of the electron transfer can be detected. In the case where a catalyst is used as the electrode reactive substance, for example, the detection method of the present invention can further use an electron transfer mediator. The electron transfer mediator mediates the transfer of electrons between the catalyst and the electrode, for example. In other words, in the case where the electron transfer mediator is used, the catalyst that has transferred electrons with a reactive substance transfers electrons with the electron transfer mediator. Then, in the case where the electron transfer mediator electrochemically reacts with the electrode, the electron transfer between the electron transfer mediator and the electrode is caused. That is, for example, in the case where the contact frequency between the catalyst and the electrode increases, the number of redox cyclings of the electron transfer mediator increases. Since the frequency of an electrode reaction is thereby increased, an increase in the detection value of the electron transfer can be detected. Further, in the case where the electron transfer mediator is used, for example, plural electrode reactions are caused by the change of the mobility of the electrode reactive substance per molecule by the redox cycling. Therefore, the detection signal of the electron transfer can be amplified, and the S/N ratio in the detection signal is increased. For example, the electron transfer mediator may be dissolved or dispersed into a solution containing a reactive substance for the catalyst or may be immobilized to the electrode. In the case where the contact frequency between the catalyst and the electrode is increased by adding the electron transfer mediator to the solution, for example, the contact frequency between the electron transfer mediator and the catalyst is increased. Since the number of redox cyclings is thereby increased, an increase in the detection value of the electron transfer can be detected. In the case where the electron transfer mediator is immobilized to the electrode, the method of immobilizing the electron transfer mediator to the electrode is not particularly limited, and a commonly used method can be used for immobilizing the electron transfer mediator to the electrode. The electron transfer mediator can be immobilized to the electrode, for example, by a method of crosslinking the functional group of the electrode surface and the functional group of the electron transfer mediator, a method of crosslinking a substance such as a thiol molecule and a polymer with which an electron transfer mediator is preliminarily modified and the electrode surface, and the like. Further, the electron transfer mediator can be immobilized to the electrode, for example, by immobilizing the probe in which the linker is modified with the electron transfer mediator to the electrode surface. By immobilizing the electron transfer mediator to the electrode surface, the contact frequency between the electron transfer mediator and the catalyst is increased when the contact frequency between the electrode and the catalyst is increased. Since the number of redox cyclings is thereby increased, an increase in the detection value of the electron transfer can be detected. Further, an operation of adding the electron transfer mediator to the solution becomes unnecessary. In the detection method of the present invention, the catalyst is not particularly limited and any catalyst can be used. The catalyst may be the enzyme as described above. For example, oxidase such as glucose oxidase and bilirubin oxidase; dehydrogenase such as glucose dehydrogenase; coenzyme oxidase such as diaphorase; peroxide reductase such as horseradish peroxidase and catalase; and metal catalysts such as Pt and titanium oxide can be used. The reactive substance for the catalyst can be selected suitably depending on the catalyst to be used, for example. Especially, in the case where the catalyst is an enzyme, the substrate can be selected suitably depending on the enzyme to be used, for example. Also, the electron transfer mediator can be selected suitably depending on the catalyst to be used, for example. Although the number of electrode reactive substances is not particularly limited, since the change of the electron transfer caused by binding or separation of one molecule of the aptamer is large, for example, the probe preferably contains more than one electrode reactive substances per molecule of the probe.

The linker is bindable to at least one of the aptamer-bindable substance and the labeling substance, and also is bindable to the support. In the present invention, in the case where the aptamer is a nucleic acid, preferably, the linker does not contain at least a part of a complementary strand of the aptamer, for example. Specifically, in the case where the linker is a nucleic acid, for example, it is more preferable that the nucleic acid does not contain the base sequence of the complementary strand of the aptamer 7 bases or more and it is yet more preferable that the nucleic acid does not contain the base sequence of the complementary strand of the aptamer 4 bases or more. When the number of bases is in this range, for example, the interaction between the linker and the aptamer that will be described below is weak, which can be ignored in the detection method of the present invention. The linker is, for example, a chain polymer or oligomer or a dendritic polymer or oligomer, and is preferably a substance that does not interact with the aptamer or a target substance. In the case where a substance that does not interact with the aptamer or a target substance is used, the nonspecific adsorption of the aptamer, the target substance, and the like can be suppressed, and the specificity of the detection method of the present invention can further be increased. Further, since the aptamer that is bound to the target substance is promptly separated from the probe, the detection speed of a target substance can further be increased. Although the foregoing polymer and oligomer are not particularly limited and both of a natural polymer and a synthetic polymer can be used, for example, a hydrophilic polymer is preferable. In the case where the hydrophilic polymer is used as the linker, for example, the nonspecific adsorption of the aptamer can be suppressed when the aptamer is a nucleic acid. Examples of the hydrophilic polymer and oligomer include polyether such as polyethylene glycol; polylactic acid; and polyacrylamide. As the linker, also, a substance having a negative charge is preferred. In the case where the linker has a negative charge, for example, the aptamer can be repulsed electrostatically when the aptamer is polyanion. Examples of the foregoing polymer and oligomer having a negative charge include nucleic acids, heparin, and polyacrylic acids. In the detection method of the present invention, especially, in the case where a nucleic acid is used as the linker, as described above, the interaction between the linker, which is the nucleic acid, and the aptamer can further be avoided when the linker does not have a base sequence that is complementary to the aptamer. The linker preferably includes a functional group for immobilizing the probe to the support at at least one end thereof. Such a functional group is not particularly limited. In the case where the support is an electrode, the functional group can be applied to the linker using a commonly used method for modifying an electrode surface, for example. Examples of the method include a method of modifying one end of the linker with a thiol group and forming a metal-sulfur bond with a metal electrode surface and a method of modifying one end of the linker with an amino group and forming an amide bond with an electrode surface that is modified with a carboxyl group. Also, a method of modifying one end of the linker with biotin and forming a biotin-avidin complex with an electrode surface modified with avidin can be employed, for example. The linker can be any linker as long as both of the aptamer-bindable substance and the labeling substance are not directly immobilized to a support and at least one of the aptamer-bindable substance and the labeling substance is immobilized to the support via the linker, for example. As the linker, a T-shaped linker and a linker that is branched into a Y-shape or an X-shape can be used, for example. However, the linker is not limited thereto. Further, the linker may be composed of plural straight chain linker molecules, for example. Specifically, for example, plural linker molecules are linked to each other via at least one of the nucleic acid aptamer-bindable substance and the labeling substance, and the plural linker molecules may be immobilized to the support via a linker molecule positioned at one end thereof. Further, for example, the labeling substance and the aptamer-bindable substance may be crosslinked directly without involving the linker, the linker may be bound to at least one of the labeling substance and the aptamer-bindable substance, and the linker may be bound to the support. The probe can be produced by binding the aptamer-bindable substance and the labeling substance to the linker by suitably using a known organic synthetic method, for example. Specifically, the probe can be produced by binding the aptamer-bindable substance, the labeling substance, and a functional group for immobilizing the linker to the support to the linker using a linking group. As the linking group, for example, DNA can be used. The DNA is preferably the one that does not interact with the aptamer or the aptamer candidate substance that will be described below, and DNA having a random base sequence, DNA having a single base sequence such as poly A, and the like can be used, for example. As the linker, for example, a dendritic structure linker that is branched into three parts can be used. As such a linker, for example, the linker composed of hydrocarbon having a dimethoxytrityl group, a 9-fluorenylmethyloxycarbonyl group, and phosphoramidite at the respective ends of the branches can be used. Specifically, for example, a commercially available linker such as “Asymmetric Doubler Phosphoramidite (trade name)” available from Glen Research can be used. Further, for example, three types of DNAs are synthesized and bound to the respective ends of the branches of the linker that is branched into three parts using a known DNA solid phase synthesis method. Furthermore, for example, a functional group for modifying the labeling substance, a functional group for modifying the aptamer-bindable substance, and a functional group for binding to the support are added to the ends of the three types of DNAs. Such a terminal functional group can be selected, for example, from a carboxyl group, an amino group, and a thiol group. However, the terminal functional group is not limited thereto. Specifically, for example, the terminal functional group can be selected suitably depending on the structures of the labeling substance and the aptamer-bindable substance and the material of the support. Then, for example, the labeling substance and the aptamer-bindable substance are modified with the terminal functional groups using an amino coupling method. The order of binding the labeling substance and the aptamer-bindable substance to the terminal functional group is not limited, for example. That is, after the labeling substance is modified with the terminal functional group, the aptamer-bindable substance may be modified with the terminal functional group; and vice versa. The probe synthesized in this manner can be immobilized to the support by the terminal functional group that has not been used. As the terminal functional group for binding the support, for example, a thiol group can be used.

In the detection method of the present invention, the aptamer is applicable as long as it is a nucleic acid or a peptide specifically bindable to a target substance. For example, the aptamer can be DNA, RNA, or PNA, which is an artificial nucleic acid. The aptamer is not particularly limited, and is preferably the one having the structure that specifically binds to an epitope of the target substance. The aptamer may have a part that does not bind to the target substance. In the case where the aptamer is a nucleic acid, the aptamer can be any nucleic acid as long as it has a sequence of an aptamer that specifically binds to an epitope of a target substance, for example. For example, the aptamer may have a base sequence that does not bind to the target substance. The aptamer is preferably the one that binds to the target substance more easily than the aptamer-bindable substance, for example. In the case where the aptamer binds to the target substance more easily than to the aptamer-bindable substance, the aptamer is easily separated from the aptamer-bindable substance to which the aptamer has been bound, for example, and a large detection signal can be obtained. Such an aptamer can be obtained using a known aptamer screening method such as the SELEX method, for example, and can be obtained by the method of screening an aptamer of the present invention that will be described below. In particular, an aptamer that is obtained using the same probe as the detection method of the present invention by the method of screening an aptamer of the present invention that will be described below is very suitable for the detection method of the present invention. In the case where the aptamer is a nucleic acid, the nucleic acid may be a single-stranded nucleic acid or a double-stranded nucleic acid. The aptamer may partially have a double-stranded nucleic acid part. In the case where the aptamer has a double-stranded nucleic acid part, for example, the double-stranded nucleic acid part can be present anywhere in the aptamer. For example, the double-stranded nucleic acid part may be present at an end of the single-stranded nucleic acid or at the middle of the single-stranded nucleic acid. Further, the whole sequence of the single-stranded nucleic acid of the aptamer may form a double-stranded part by hybridizing to a nucleic acid fragment that has been prepared separately. However, since the double-stranded nucleic acid part easily causes a branch migration at the time of binding the target substance with the aptamer, the double-stranded nucleic acid part is preferably formed so as to have a part of a base sequence that binds to the target substance. In the detection method of the present invention, an aptamer that is recovered by the method of screening an aptamer of the present invention that will be described below can be used. In the screening method, in the case where the aptamer is recovered with the same probe as that used in the detection method of the present invention, the aptamer recovered shows a high degree of selectivity in the detection method of the present invention and is preferred.

In the detection method of the present invention, the support is not particularly limited as long as it can immobilize the aptamer-bindable substance and the labeling substance via the linker, for example. For example, an electrode can be used as the support. The electrode is not particularly limited as long as it has electrical conductivity. As the electrode, specifically, gold, platinum, and carbon can be used preferably because they have high electrical conductivity and have the surfaces that can be modified easily, for example. The shape of the electrode is not particularly limited as long as the detection method of the present invention can be performed, for example. Examples of the shape include a disk, a flat plate, a thin film, and a particle, and can be selected appropriately depending on the needed detection sensitivity, reliability, and the like. In the case where the electrode is a particle, since a specific surface area of the electrode can be enlarged, the detection sensitivity can be increased. In the case where the electrode has a disk shape, a flat plate shape, or a thin film shape, for example, since variations in size of electrode area can be suppressed, the reproducibility of the target substance can be improved. The surface of the electrode may be treated so that the nonspecific adsorption of the aptamer can be suppressed. However, the surface treatment is preferably performed such that the probe is exposed to the electrode surface even after the surface treatment. The specificity of a target substance can be increased by such a surface treatment, for example. As the surface treatment, a commonly used method of preventing nonspecific adsorption to an electrode can be used, and an appropriate method can be selected depending on the properties such as the size and the structure of the probe to be used. An example of the size of the probe includes a molecular weight and an example of the structure includes a molecular structure. The surface treatment can be performed, for example, by applying hydrophilic molecules such as dextrans, ethylene glycol, ethylenimine, ethylene oxide, and polymers thereof, or the hydrophilic molecules and thiols having a molecular structure in which hydrophilic functional groups such as a carboxyl group and a hydroxyl group are exposed on the surfaces of the thiol molecule, and proteins such as albumin on the surfaces to the electrode.

In the detection method of the present invention, the separation detecting unit that detects separation of the aptamer in the detection step (B) is, for example, a device that can detect a signal produced by the labeling substance. In the case where the labeling substance produces an optical signal, an electrical signal, or a color signal, for example, the separation detecting unit is, for example, a device that can detect the change in the optical signal, the electrical signal, or the color signal that is produced by the labeling substance before and after separation of the aptamer from the aptamer-bindable substance. Especially, in the case where the electrode reactive substance is used as the labeling substance, an electron transfer detecting unit can be used as the separation detecting unit, for example. The electron transfer detecting unit is not particularly limited, and is, for example, a device for electrochemically detecting the electron transfer. Such electrochemical detection can be performed by any method as long as the electron transfer between the electrode reactive substance and the electrode can be detected, for example. The electrochemical detection may be a method of detecting the change in current value, voltage, impedance, or the like, for example, by using the cyclic voltammetry method, the differential pulse voltammetry method, the square wave voltammetry method, the alternating current voltammetry method, the alternating current impedance method, the chronoamperometry method, the chronopotentiometry method, and the like. The larger the amount of the electron transfer is, the higher the absolute value of the current. The larger the amount of the electron transfer is, the lower the absolute value of the voltage. Further, the larger the amount of the electron transfer is, the lower the impedance. In the detection method of the present invention, the electrochemical measurement is preferably performed as follows, for example. That is, in the case where the electrode reactive substance is the substance having an oxidation-reduction potential, the electrochemical measurement is preferably performed under the condition in which the substance having an oxidation-reduction potential electrochemically reacts with the electrode. Further, in the case where the electrode reactive substance is a catalyst, the electrochemical measurement is preferably performed under the condition in which the catalyst electrochemically reacts with the electrode in a solution containing a reactive substance for the catalyst, for example. Furthermore, in the case where the electrode reactive substance is a catalyst, the electrochemical measurement is preferably performed under the condition in which the electron transfer mediator electrochemically reacts with the electrode in a solution further containing the electron transfer mediator, for example.

[Target Substance Detection Apparatus]

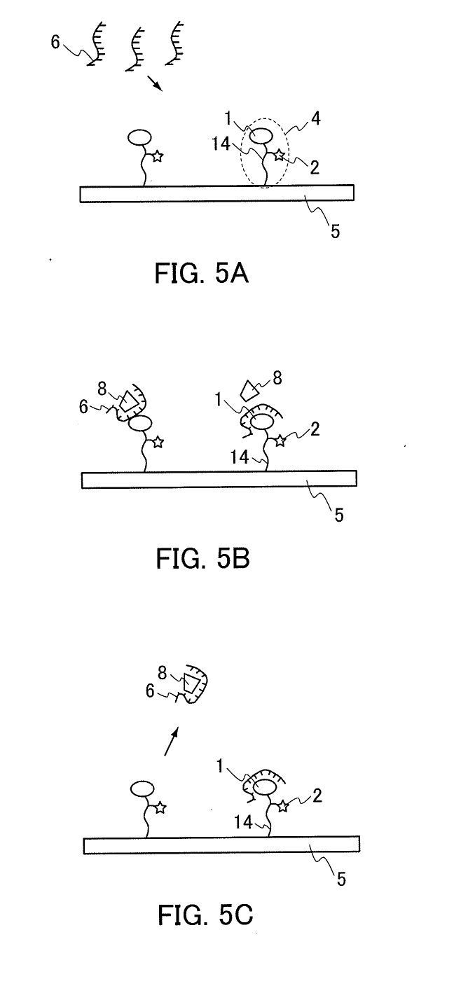

Next, the target substance detection apparatus of the present invention will be described. As described above, the target substance detection apparatus of the present invention includes a probe in which an aptamer-bindable substance and the labeling substance are each bound to a linker immobilizable to a support, a separation detecting unit for detecting separation of the aptamer from the aptamer-bindable substance due to binding between a target substance in a sample and the aptamer based on the labeling substance, and a support that immobilizes the probe via the linker at the time of detecting the separation. FIG. 5A shows an example of the configuration of the target substance detection apparatus of the present invention. As shown in FIG. 5A, the target substance detection apparatus includes a probe 4 in which an aptamer-bindable substance 1 and a labeling substance 2 are each bound to a linker 14, a support 5 for immobilizing the probe 4 via the linker 14, and a separation detecting unit, which is not illustrated. The separation detecting unit is not particularly limited, and is, for example, as follows. The probe 4 is applicable as long as it is immobilized to the support 5 via the linker 14 at the time of detecting a target substance, for example. Such a configuration makes it possible to detect separation of the aptamer from the aptamer-bindable substance 1 due to binding between a target substance in a sample and the aptamer by the separation detecting unit. In the target substance detection apparatus of the present invention, first, the aptamer is bound to the aptamer-bindable substance of the probe at the time of detecting a target substance. Binding of the aptamer to the aptamer-bindable substance can be achieved, for example, by adding the aptamer to the aptamer-bindable substance of the probe at the time of detecting the target substance. Further, for example, the probe in which the aptamer is bound to the aptamer-bindable substance may be purchased preliminarily. Then, for example, a sample is added to the aptamer-bindable substance to which the aptamer is bound. Thereby, in the case where the sample contains the target substance, the aptamer that is bound to the aptamer-bindable substance binds to the target substance and is separated from the aptamer-bindable substance. In the target substance detection apparatus of the present invention, separation of the aptamer from the aptamer-bindable substance is detected by detecting a target substance by the separation detecting unit. It is to be noted that the target substance detection apparatus of the present invention can be used for the method of screening an aptamer of the present invention that will be described below.

The mechanism of the detection of a target substance by the target substance detection apparatus of the present invention can be explained as follows, for example. That is, in the target substance detection apparatus of the present invention, the dynamic behavior of the probe in a reaction phase changes between the case in which the target substance binds to the aptamer and the aptamer is separated from the aptamer-bindable substance and the case in which the sample does not contain the target substance and the aptamer is kept bound to the aptamer-bindable substance. In the target substance detection apparatus of the present invention, since the probe includes the labeling substance and the probe is immobilized to the support at the time of detecting a target substance, for example, the change of the dynamic behavior of the probe shown by the labeling substance can be detected easily by the separation detecting unit, and a target substance can be detected easily.

In the target substance detection apparatus of the present invention, as the probe, for example, the same probe as that used in the detection method of the present invention can be used. The structure and function of the probe are as described above. The probe is applicable as long as it is immobilized to the support at the time of detecting a target substance. For example, at the time of detecting a target substance, in the target substance detection apparatus of the present invention, in the case where the aptamer is bound to the aptamer-bindable substance, the probe can be immobilized to the support before, during, or after the step of binding the aptamer to the aptamer-bindable substance, for example.

In the target substance detection apparatus of the present invention, as the separation detecting unit, for example, a device that can detect a signal produced by the labeling substance can be used.

In the target substance detection apparatus of the present invention, as the support, for example, the same support as that used in the detection method of the present invention can be used. As the support, for example, an electrode can be used. Thereby, by immobilizing the probe to an electrode, the target substance can be detected using the separation detecting unit that detects separation of the aptamer from the probe as an electrical reaction using the electrode. In other words, in this case, the change of the dynamic behavior of the probe in a reaction phase between the case in which the target substance binds to the aptamer and the aptamer is separated from the probe and the case in which the aptamer is kept bound to the probe can be detected by the separation detecting unit as the electrical reaction between the labeling substance and the electrode. The electrode is not particularly limited, and is similar to the electrode described in the explanation of the detection method of the present invention. The structure and function of the electrode are as described above.

In the target substance detection apparatus of the present invention, especially, in the case where an electrode reactive substance is used as the labeling substance, the separation detecting unit preferably includes an electron transfer detecting unit that detects the electron transfer between the electrode reactive substance and the electrode. In this case, separation of the aptamer from the aptamer-bindable substance can be detected, for example, as follows. That is, at the time of detecting a target substance, the probe is immobilized to an electrode. Further, in the separation detecting unit, separation of the aptamer from the probe can be detected, for example, by comparing the detection value of the electron transfer between the electrode reactive substance and the electrode prior to supply of the target substance to the probe and the detection value of the electron transfer between the electrode reactive substance and the electrode after supply of the target substance to the probe. The electron transfer detecting unit is not particularly limited and is similar to the electron transfer detecting unit described in the explanation of the detection method of the present invention, for example. The structure and function of the electron transfer detecting unit are as described above. According to this embodiment, since the probe includes the electrode reactive substance, for example, there is no need to modify the aptamer with the electrode reactive substance. Further, since the aptamer can be immobilized to the electrode by the probe, for example, there is no need to modify the aptamer with a functional group for formation of a crosslinking reaction with an electrode surface. Accordingly, a target substance can be detected very simply. Further, the target substance detection apparatus of this embodiment shows a signal-increasing type detection reaction in which the detection value of the electron transfer increases as the target substance increases, and can achieve a high S/N ratio. The detection mechanism of a target substance in this embodiment can be described as follows, for example. However, the following descriptions are mere illustrations and do not limit the present invention. When the target substance binds to the aptamer that has been bound to the aptamer-bindable substance and the aptamer is separated from the aptamer-bindable substance, the mobility of the probe in a reaction phase increases. On the other hand, for example, in the case where the target substance is not a target substance to which the aptamer is bindable, since the aptamer bound to the aptamer-bindable substance does not bind to the target substance, the aptamer is not separated from the aptamer-bindable substance. Therefore, the mobility of the probe in the reaction phase remains low. Here, in the target substance detection apparatus of the present invention in this embodiment, the probe is immobilized to the electrode via the linker. Therefore, in the case where the mobility of the probe is high, the contact frequency between the electrode reactive substance in the probe and the electrode is high. On the other hand, in the case where the mobility of the probe is low, the contact frequency between the electrode reactive substance in the probe and the electrode is low. Further, the higher the contact frequency between the electrode reactive substance and the electrode is, the higher the detection value of the electron transfer between the electrode reactive substance and the electrode. In other words, the detection value of the electron transfer in the case where the aptamer is not bound to the aptamer-bindable substance is higher than that in the case where the aptamer is bound to the aptamer-bindable substance. The target substance can be detected by detecting the change of the electron transfer.

The target substance detection apparatus of the present invention may further include a binding state detection unit for detecting the state in which the aptamer is bound to the aptamer-bindable substance of the probe. Thereby, for example, the target substance can be detected by comparing the detection result obtained by the binding state detection unit and the detection result obtained by the separation detecting unit. The binding state detection unit may have a configuration similar to that of the separation detecting unit, for example. The binding state detection unit may be the same device as the separation detecting unit or may be a different device from the separation detecting unit.

The target substance detection apparatus of the present invention may further include an unbound aptamer removing unit that removes the aptamer that is not bound to the aptamer-bindable substance. Thereby, the target substance can be prevented from binding to the aptamer that is not bound to the aptamer-bindable substance. As a result, the reproducibility of the amount of the aptamer that is separated from the aptamer-bindable substance can be improved and a detection result that reflects the presence of the target substance more correctly can be obtained.

With respect to the target substance detection apparatus of the present invention, for example, the probe can be used in a liquid such as a solution. The liquid is not particularly limited, and is similar to the liquid described in the explanation of the detection method of the present invention, for example. The composition, conditions, and the like of the liquid are as described in the explanation of the detection method of the present invention.

The target substance detection apparatus of the present invention may further include the aptamer. As described above, the aptamer is applicable as long as it binds to the aptamer-bindable substance at the time of detecting a target substance. The aptamer is not particularly limited, and is similar to the aptamer described in the explanation of the detection method of the present invention, for example. The structure and function of the aptamer are as described above.

In the target substance detection apparatus of the present invention, for example, the target substance can be detected by using a known detection value as the detection value of the electron transfer between the electrode reactive substance and the electrode prior to supply of the target substance to the probe, and comparing the known detection value and the detection value of the electron transfer between the electrode reactive substance and the electrode after supply of the target substance to the probe. The known detection value can be obtained preliminarily by conducting an experiment separately, for example. Further, the known detection value can be calculated, for example, from the density of the probe on the electrode prior to supply of the target substance and the ratio between the number of probes that are bound to the aptamers and the number of probes that are not bound to the aptamers. That is, thereby, the detection value of the electron transfer between the electrode reactive substance and the electrode can be preliminarily obtained using an electrode to which the probe and the aptamer are bound under known conditions. As a result, the target substance can be detected from the difference between the known detection value and the detection value of the electron transfer in the state where the aptamer is separated.

[Method of Screening Aptamer]

Next, the method of screening an aptamer of the present invention will be described. In the screening method of the present invention, first, in the first step (hereinafter, this may also be referred to as the “first step (A′)”), the aptamer candidate substance is supplied to the probe to bind the aptamer candidate substance to the probe. Then, in the second step (hereinafter, this may also be referred to as the “second step (B′)”), the target substance is supplied to the probe to which the aptamer candidate substance has been supplied. Thereby, in the case where the aptamer candidate substance is an aptamer bindable to the target substance, the aptamer candidate substance that has been bound to the probe in the first step (A′) binds to the target substance and is separated from the probe. Subsequently, in the third step (hereinafter, this may also be referred to as the “third step (C′)”), the aptamer candidate substance that has been separated in the second step (B′) is recovered. In other words, in the case where the aptamer candidate substance is not an aptamer of the target substance, in the second step (B′), the aptamer candidate substance is kept bound to the probe and is not separated from the probe. Here, in the screening method of the present invention, the probe is immobilized to a support via the linker in the second step. Accordingly, the aptamer candidate substance separated from the probe due to binding between the target substance and the aptamer in the second step (B′) can be recovered easily from the aptamer candidate substance that does not bind to the target substance and is kept bound to the probe. In this manner, according to the screening method of the present invention, an aptamer of the target substance can be obtained efficiently. Further, the aptamer recovered in this manner has a high binding ability to the target substance and is very suitable for detection of the target substance. Furthermore, since the probe includes a labeling substance, binding of the aptamer candidate substance to the aptamer-bindable substance and separation of the aptamer candidate substance from the aptamer-bindable substance can be detected based on the labeling substance, and the separation status of the aptamer candidate substance can be monitored. The monitoring of the separation status of the aptamer candidate substance will be described below.

The screening method of the present invention is performed, for example, in a liquid such as a solution. The liquid is not particularly limited as long as binding of the aptamer candidate substance to the probe and separation of the aptamer candidate substance from the probe occur, for example. In other words, the conditions such as the composition of the liquid for performing the screening method, a temperature, a pH, and an electrolyte in each of the steps of the screening method are not particularly limited as long as the binding of the aptamer candidate substance to the probe and separation of the aptamer candidate substance from the probe occur, for example. As the conditions, for example, conditions commonly used for the SELEX method can be used, and the conditions can be set suitably so that the binding of the aptamer candidate substance to the probe and separation of the aptamer candidate substance from the probe occur. For example, by equalizing the conditions of the liquid used in the screening method of the present invention and the conditions of the liquid used in the detection method of the present invention, an aptamer having a high degree of selectivity that is very suitable for the detection method of the present invention can be recovered.

In the screening method of the present invention, the probe is not particularly limited, and can be any probe as long as an aptamer-bindable substance and a labeling substance are each bound to a linker immobilizable to a support. As the probe in which the aptamer-bindable substance and the labeling substance are each bound to the linker, the probe used in the detection method of the present invention can be used. In the case where the probe used in the detection method of the present invention is used, a probe that is very suitable for the detection method of the present invention can be recovered. The configuration and function of such a probe are as described in the explanation of the detection method of the present invention. Especially, in the case where a probe including the double-stranded nucleic acid aptamer-bindable substance is used as the probe, the aptamer bound to the target substance can be recovered without immobilizing a substance having an epitope that is identical to or similar to the whole or a part of the target substance to a support such as a substrate, or the like. Therefore, in this case, especially, the screening method of the present invention can be utilized, for example, for obtaining various aptamers that contain double-stranded sequence parts, and therefore general versatility can be increased. In other words, for example, also with respect to a substance that does not have a functional group that can be used for a reaction for immobilizing to a support such as a substrate, a substance having a high degradability, and a substance whose aptamer could hardly be obtained because the epitope could not be immobilized to the support, the aptamer can be obtained. Further, for example, also with respect to a substance in which the epitope has conventionally been vanished or the epitope has conventionally been hidden due to immobilization to the support, the aptamer can be obtained.

In the screening method of the present invention, the aptamer candidate substance to be bound to the aptamer-bindable substance is not particularly limited, and many types of the aptamer candidate substances are preferably provided. Use of various aptamer candidate substances makes it possible to recover the aptamer having a higher binding ability to the target substance, for example. Such aptamer candidate substances can be produced, for example, by the SELEX method or the like. According to the method of screening an aptamer of the present invention, for example, an aptamer capable of detecting a target substance without being modified with an electrode reactive substance, a functional group, or the like can be recovered. Even in the case where various aptamer candidate substances are used, an aptamer that is suitable for detection of a target substance can be recovered efficiently.

In the screening method of the present invention, recovery of the aptamer in the third step (C′) can be performed using any unit that is capable of recovering the aptamer candidate substance that binds to the target substance and is separated from the probe by screening from the aptamer candidate substance that does not bind to the target substance and is kept bound to the probe, for example. The aptamer candidate substance can be recovered by pouring a liquid onto the surface of the support, for example.

In the screening method of the present invention, for example, the aptamer candidate substance that has been recovered in the third step (C′) may be supplied as the aptamer candidate substance of the first step (A′), and the procedure from the first step (A′) to the third step (C′) can be performed repeatedly. Thereby, for example, the purity of the aptamer of the target substance in the recovered substance recovered in the third step (C′) can be increased. The screening method of the present invention may further include the fourth step (hereinafter, this may also be referred to as the “fourth step (D′)”) of amplifying the aptamer candidate substance that has been recovered in the third step (C′), for example. In the screening method of the present invention, for example, further, the aptamer candidate substance amplified in the fourth step (D′) may be supplied as the aptamer candidate substance in the first step (A′), and the procedure from the first step (A′) to the third step (C′) or the procedure from the first step (A′) to the fourth step (D′) may be performed repeatedly. Thereby, for example, a large quantity of high-performance aptamer can be obtained. In the case where the procedure is performed repeatedly, in the screening method of the present invention, for example, another aptamer candidate substance can be supplied to the aptamer candidate substance as the aptamer candidate substance in the first step. “Another aptamer candidate substance” refers to an aptamer candidate substance that is different from the aptamer candidate substance recovered in the third step (C′), for example. For example, in the case where the aptamer candidate substance recovered in the third step (C′) is a nucleic acid, “another aptamer candidate substance” is, for example, a mixture of nucleic acids having various sequences (this is also referred to as a nucleic acid pool). Thereby, for example, a higher-performance aptamer can be recovered. The nucleic acid pool can be added as the aptamer candidate substance by amplifying a nucleic acid by a method in which an error occurs in a nucleic acid amplification step of an aptamer candidate substance by PCR or by adding a nucleic acid mixture synthesized separately to the aptamer candidate substance that has been amplified in the nucleic acid amplification step, for example.

The screening method of the present invention may further include a fifth step of removing the aptamer candidate substance that is not bound to the probe prior to the second step (B′), for example. Thereby, an aptamer candidate substance that is different from the aptamer of the target substance can be prevented from being mixed in the aptamer candidate substance recovered in the third step (C′), and the accuracy of recovery of aptamer can be increased. The screening method of the present invention may further include a sixth step of removing the aptamer candidate substance that is not bound to the target substance in the third step (C′), for example. Thereby, the unintended aptamer candidate substance that is not bound to the target substance can be removed.

The screening method of the present invention may further include a step of removing the aptamer candidate substance that is bound to the target substance analog after the aptamer candidate substance that is bound to the probe is brought into contact with a target substance analog having a structure similar to the target substance prior to the second step (B′), for example. Thereby, for example, the aptamer candidate substance that is bound to the analog can be removed, and the aptamer candidate substance having a high degree of specificity to the target substance can be recovered.

As described above, in the screening method of the present invention, the probe includes a labeling substance. Therefore, for example, the detection step in which separation of an aptamer candidate substance in the second step is detected based on the labeling substance can be performed. In other words, for example, the change of the dynamic behavior of the probe in a reaction phase with binding of the aptamer candidate substance to the probe and separation of the aptamer candidate substance from the probe can be detected based on the labeling substance, and the separation status of the aptamer candidate substance can be monitored. The detection can be performed as long as the probe is immobilized to the support at the time of the detection step, for example. In other words, the probe can be immobilized to the support before, during, or after the step of binding the aptamer candidate substance to the probe, for example. As the support, for example, the support used in the detection method of the present invention can be used. The configuration and function of such a support are as described in the explanation of the detection method of the present invention. As the support, for example, an electrode can be used. Thereby, separation of the aptamer candidate substance in the second step can be detected as the electrical reaction between the labeling substance and the electrode. As the electrode, for example, the same electrode as that explained in the detection method of the present invention can be used.

In the screening method of the present invention, the probe including an electrode reactive substance as the labeling substance can be used, for example. In this case, for example, binding of the aptamer candidate substance to the probe and separation of the aptamer candidate substance from the probe can be detected by immobilizing the probe to an electrode. The detection can be performed by comparing the detection value of the electron transfer between the electrode reactive substance and the electrode prior to supply of the target substance to the probe and the detection value of the electron transfer between the electrode reactive substance and the electrode after supply of the target substance to the probe. For example, a known detection value may be used as the detection value of the electron transfer between the electrode reactive substance and the electrode prior to supply of the target substance to the probe, and the known detection value may be compared with the detection value of the electron transfer between the electrode reactive substance and the electrode after supply of the target substance to the probe. This makes it possible to detect the binding state in which the aptamer candidate substance binds to the aptamer-bindable substance and the separation state in which the aptamer candidate substance is separated from the aptamer-bindable substance during the screening method, and it can be detected immediately whether the aptamer candidate substance is separated, for example. Further, for example, by setting the amount of separation of the intended aptamer, an appropriate timing such as the termination point of the first step (A′) or the second step (B′) or the starting point or termination point of the third step (C′) can be decided with the change of the electron transfer as an indicator, for example. In the case where the procedure from the first step (A′) to the fourth step (D′) is performed repeatedly, for example, the termination point thereof can also be decided. Further, it can be checked on the spot whether the conditions such as composition, a pH, and a temperature of the solution used in each of the aforementioned steps are appropriate. Therefore, the efficiency and productivity of a recovery operation of aptamer can be increased. The detection value of the electron transfer between the electrode reactive substance and the electrode can be obtained, for example, by using the electron transfer detecting unit that is used in the detection method of the present invention.

[Aptamer Screening Apparatus]

Next, the aptamer screening apparatus of the present invention will be described. As described above, the aptamer screening apparatus of the present invention includes a probe in which an aptamer-bindable substance and a labeling substance are each bound to a linker immobilizable to a support, a recovering unit for recovering the aptamer candidate substance separated from the probe due to binding between a target substance and the aptamer, and a support that immobilizes the probe. FIG. 5A shows an example of the configuration of the aptamer screening apparatus of the present invention. As shown in FIG. 5A, the aptamer screening apparatus includes a probe 4 that specifically binds to an aptamer candidate substance 6, a recovering unit, which is not illustrated, and a support 5. The recovering unit recovers the aptamer candidate substance 6 that is separated from the aptamer-bindable substance 1 due to binding between a target substance and the aptamer. According to the aptamer screening apparatus of the present invention, for example, an aptamer can be recovered as follows. That is, first, the aptamer candidate substance is bound to the probe by adding an aptamer candidate substance to the probe. Then, the target substance is added to the probe to which the aptamer candidate substance has been added. Thereby, in the case where the aptamer candidate substance is an aptamer that is bindable to the target substance, for example, the aptamer candidate substance that is bound to the probe binds to the target substance and is separated from the probe. In the case where the aptamer candidate substance is not the aptamer of the target substance, for example, the aptamer candidate substance is kept bound to the probe and is not separated from the probe. Here, the probe is immobilized to the support at the time of separation of the aptamer candidate substance, for example. Accordingly, for example, the aptamer candidate substance that is separated from the probe due to binding between the target substance and the aptamer can be screened easily from the aptamer that does not bind to the target substance and is kept bound to the probe, and the aptamer candidate substance can be recovered using the recovering unit. In this manner, according to the aptamer screening apparatus of the present invention, for example, an aptamer of the target substance can be obtained efficiently. Further, for example, the aptamer recovered in this manner has a high binding ability to the target substance, and is very suitable for detection of the target substance. According to the aptamer screening apparatus of the present invention, for example, an aptamer capable of detecting a target substance can be recovered without being modified with an electrode reactive substance, a functional group, or the like. Further, since the probe includes a labeling substance, for example, binding of the aptamer candidate substance to the aptamer-bindable substance and separation of the aptamer candidate substance from the aptamer-bindable substance can be detected based on the labeling substance, and the separation status of the aptamer candidate substance can be monitored. The monitoring of the separation status of the aptamer candidate substance will be described below.

With respect to the aptamer screening apparatus of the present invention, for example, the probe can be used in a liquid such as a solution. The composition, conditions, and the like of the liquid are the same as those of the liquid that is used in the screening method of the present invention, for example.

In the aptamer screening apparatus of the present invention, the probe is not particularly limited, and can be any probe as long as an aptamer-bindable substance and a labeling substance are each bound to a linker that is immobilizable to a support. In the case where the probe that is used in the detection method of the present invention is especially used as the probe, for example, an aptamer that is very suitable for the use in the detection method of the present invention can be obtained. As the probe in which the aptamer-bindable substance and the labeling substance are each bound to the linker, for example, the probe used in the detection method of the present invention can be used. In the case where the probe used in the detection method of the present invention is used, for example, a probe that is very suitable for the detection method of the present invention can be recovered. The configuration and function of the probe in which the aptamer-bindable substance and the labeling substance are each bound to the linker are as described in the explanation of the detection method of the present invention, for example. Especially, in the case where a probe including the double-stranded nucleic acid aptamer-bindable substance is used as the probe, for example, the aptamer bound to the target substance can be recovered without immobilizing a substance having an epitope that is identical to or similar to the whole or a part of the target substance to an electrode or the like. Therefore, in this case, especially, the screening method of the present invention can be utilized, for example, for obtaining various aptamers that contain double-stranded sequence parts, and therefore general versatility can be increased. In other words, for example, also with respect to a substance that does not have a functional group that can be used for a reaction for immobilizing to a support such as a substrate, a substance having a high degradability, and a substance whose aptamer could hardly be obtained because the epitope could not be immobilized to the support, the aptamer can be obtained. Further, for example, also with respect to a substance in which the epitope has conventionally been vanished or the epitope has conventionally been hidden due to immobilization to the support, the aptamer can be obtained.

In the aptamer screening apparatus of the present invention, the recovering unit is not particularly limited as long as the aptamer candidate substance separated from the probe can be screened and recovered from the aptamer candidate substance that does not bind to the target substance and is kept bound to the probe. The recovering unit may be the liquid used in the case of recovering the aptamer separated from the aptamer-bindable substance by pouring a liquid onto the surface of the support, for example.

The aptamer screening apparatus of the present invention may further include an adding unit for adding the aptamer candidate substance recovered by the recovering unit to the probe, for example. Thereby, for example, the procedure in which the aptamer candidate substance is separated from the probe by adding the target substance after the aptamer candidate substance is bound to the probe and the aptamer of the target substance is recovered can be performed repeatedly. As a result, for example, the purity of an aptamer of the target substance in the recovered substance recovered by the recovering unit can be increased.

The aptamer screening apparatus of the present invention may further include an amplifying unit for amplifying the aptamer candidate substance recovered by the recovering unit and an amplified aptamer adding unit for adding the aptamer candidate substance amplified by the amplifying unit to the probe, for example. The amplifying unit is not particularly limited as long as it can amplify the aptamer candidate substance. The amplified aptamer adding unit is not particularly limited as long as it can add the aptamer candidate substance amplified by the amplifying unit to the probe. The amplified aptamer adding unit may be the same device as the adding unit, for example. Thereby, the procedure in which the aptamer candidate substance is separated by adding the target substance after the aptamer candidate substance is bound to the probe and the aptamer candidate substance of the target substance is recovered can be performed repeatedly by amplifying the aptamer candidate substance appropriately. As a result, for example, a large quantity of high-performance aptamer can be obtained. In the case where the procedure is performed repeatedly, the adding unit or the aptamer adding unit can add a mixture obtained by adding another aptamer candidate substance to the aptamer candidate substance recovered by the recovering unit to the probe, for example.

The aptamer screening apparatus of the present invention may further include an unbound aptamer removing unit for removing the aptamer candidate substance that is not bound to the probe, for example. Thereby, for example, an aptamer candidate substance that is different from the aptamer of the target substance can be prevented from being mixed in the aptamer recovered by the recovering unit, and the accuracy of recovery of aptamer can be increased.

The aptamer screening apparatus of the present invention may further include a target substance-unbound aptamer candidate substance removing unit for removing the aptamer candidate substance that is not bound to the target substance, for example. Thereby, the aptamer candidate substance that is not bound to the target substance can be removed.

In the aptamer screening apparatus of the present invention, as the support, for example, the support that is used in the method of detecting a target substance of the present invention can be used. The configuration and function of such a support are as described in the explanation of the detection method of the present invention, for example. The aptamer screening apparatus of the present invention may further include a separation detecting unit for detecting separation of the aptamer candidate substance from the probe based on the labeling substance, for example. Thereby, in a recovery operation of an aptamer candidate substance, for example, the change of the dynamic behavior of the probe in a reaction phase with separation of the aptamer candidate substance can be detected based on the labeling substance, and the separation status of the aptamer candidate substance can be monitored. For the detection, the probe is applicable as long as it is immobilized to the support at the time of detecting separation of the aptamer candidate substance from the probe, for example. In other words, the probe can be immobilized to the support before, during, or after the step of binding the aptamer candidate substance to the probe.

In the aptamer screening apparatus of the present invention, for example, as the probe, the probe including an electrode reactive substance as the labeling substance may be used. In this case, as the support, for example, an electrode can be used. Further, as the separation detecting unit, for example, an electron transfer detecting unit for detecting separation of the aptamer candidate substance from the probe due to binding between the target substance and the aptamer by detecting the electron transfer between the electrode reactive substance and the electrode can be used. As the electrode, for example, the same electrode as that described in the explanation of the detection method of the present invention can be used. Thereby, separation of the aptamer candidate substance from the probe can be detected by comparing the detection value of the electron transfer between the electrode reactive substance and the electrode prior to supply of the target substance to the probe and the detection value of the electron transfer between the electrode reactive substance and the electrode after supply of the target substance to the probe, for example. In this manner, the binding state and separation state can be detected during the screening method, and it can be detected immediately whether the aptamer candidate substance is separated, for example. Further, for example, by setting the amount of separation of the intended aptamer, an appropriate timing such as the termination point of the step of binding the aptamer candidate substance to the probe, the step of separating the aptamer candidate substance from the probe, or the like or the starting point or termination point of the step of recovering the aptamer candidate substance separated from the probe can be decided with the change of the electron transfer as an indicator, for example. Further, for example, it can be checked on the spot whether the conditions such as composition, a pH, and a temperature of the solution used for recovering an aptamer are appropriate. Therefore, for example, the efficiency and productivity of a recovery operation of aptamer can be increased. As the electron transfer detecting unit, for example, the same electron transfer detecting unit as that described in the explanation of the detection method of the present invention can be used.

Next, the Embodiments of the present invention will be described by referring to the figures. However, the following Embodiments are mere illustrations and the present invention is not limited or restricted by the following Embodiments.

### Embodiment 1

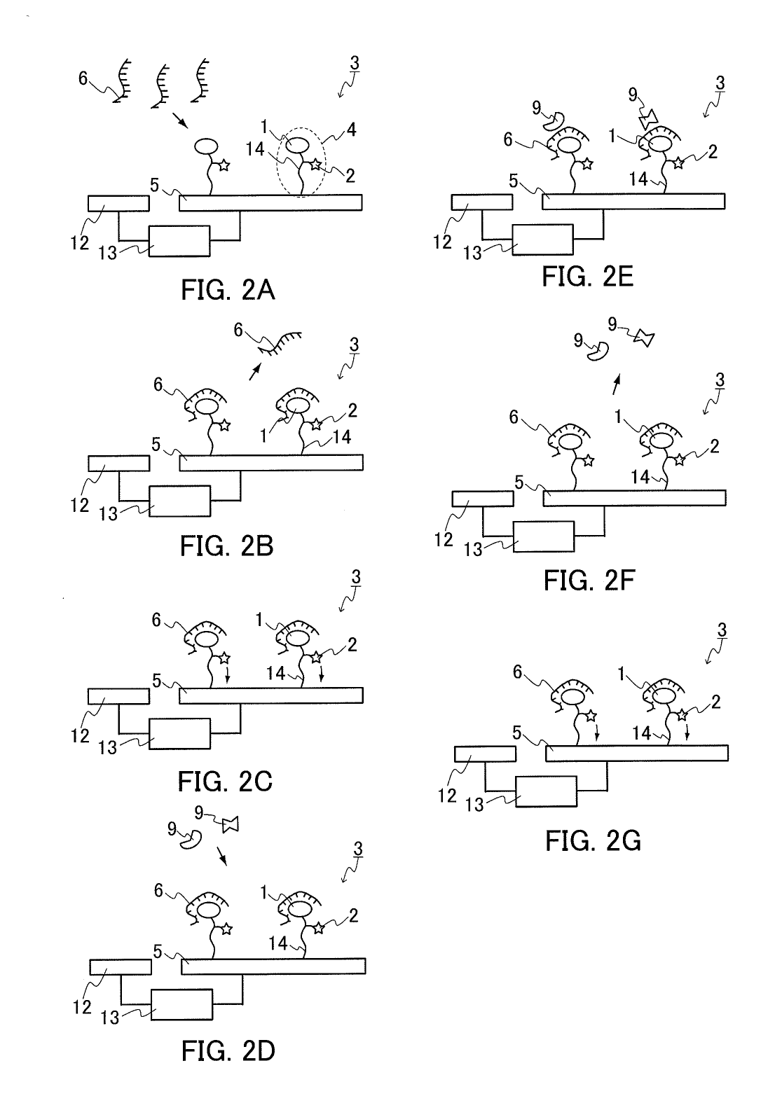

This Embodiment shows an example of the probe, the method of detecting a target substance, and the target substance detection apparatus of the present invention. The method of detecting a target substance of this Embodiment can be performed using the probe and the target substance detection apparatus of this Embodiment shown in FIGS. 1A and 2A. As shown in FIGS. 1A and 2A, a target substance detection apparatus 3 of this Embodiment includes a probe 4, an electrode 5, and an electrochemical measurement device (electron transfer detecting unit) 13 of this Embodiment. In this Embodiment, the probe 4 has the structure in which an aptamer-bindable substance 1 that is specifically bound to an aptamer 6 and an electrode reactive substance 2 are each bound to a linker 14 that is immobilizable to an electrode 5, which is a support. The linker 14 is immobilized to the electrode 5 and whereby the aptamer-bindable substance 1 and the electrode reactive substance 2 are immobilized to the electrode 5. At least a part of the aptamer 6 binds to the aptamer-bindable substance 1 of the probe 4. In this Embodiment, for example, the aptamer 6 may be a nucleic acid or a peptide. For example, the aptamer-bindable substance 1 has an epitope that is identical to or similar to the whole or a part of a target substance 8 of this Embodiment, and the epitope specifically binds to the aptamer 6. Examples of the electrode reactive substance 2 include a substance having an oxidation-reduction potential and a catalyst. The linker 14 may include at least one of a hydrophilic polymer and a hydrophilic oligomer, for example. Further, for example, the linker 14 may have a negative charge. The electrode 5 is connected to the electrochemical measurement device 13 with a counter electrode 12. The electrode 5 is a member having conductivity. In the target substance detection apparatus 3 of this Embodiment, the aptamer 6 is separated from the aptamer-bindable substance 1 due to specific binding between the aptamer 6 and the target substance 8. Here, at the time of detecting the target substance, the probe 4 is immobilized to the electrode 5 via the linker 14. Then, the electron transfer detecting unit 13 measures the electron transfer between the electrode reactive substance 2 and the electrode 5. Thereby, the aptamer binding state in which the aptamer is bound to the aptamer-bindable substance 1 and the aptamer separation state in which the aptamer 6 is separated from the aptamer-bindable substance 1 can be detected. As a result, the target substance 8 can be detected by comparing the detection value of the electron transfer between the aptamer binding state and the aptamer separation state.

Hereinafter, the detection method of this Embodiment will be described. The detection method of this Embodiment includes a probe provision step (A) and a detection step (B). Specifically, in this Embodiment, first, the probe provision step (A) and the following aptamer binding state detection step (A-2) are performed. Then, in the detection step (B), the following steps (B-1), (B-2), and (B-3) are performed.

(A) a probe provision step of providing a probe in which the aptamer 6 is bound to the aptamer-bindable substance 1.
(A-2) an aptamer binding state detection step of detecting the binding state in which the aptamer 6 is bound to the aptamer-bindable substance 1 by measuring the electron transfer between the electrode reactive substance 2 and the electrode 5 by the electron transfer detecting unit 13.
(B-1) a separation step of separating the aptamer 6 from the aptamer-bindable substance 1 by binding the target substance 8 to the aptamer 6 in the state where the aptamer 6 is bound to the aptamer-bindable substance 1.
(B-2) an aptamer separation state detection step of detecting the separation state in which the aptamer 6 is separated from the aptamer-bindable substance 1 by detecting the electron transfer between the electrode reactive substance 2 and the electrode 5 by the electron transfer detecting unit 13.
(B-3) a target substance detection step of detecting the target substance by comparing the detection value of the electron transfer detected in the aptamer binding state detection step (A-2) and the detection value of the electron transfer detected in the aptamer separation state detection step (B-2).

FIGS. 1 and 2 show the mechanism of detecting a target substance by the detection method and the target substance detection apparatus of this Embodiment. FIG. 1 shows an example in which the target substance 8 is added to the solution that will be described below. FIG. 2 shows an example that is similar to the example shown in FIG. 1 except that a non-target substance 9 is added instead of the target substance 8. Here, in this Embodiment, the following steps are performed in a solution. The composition of the solution is not particularly limited as long as binding of the aptamer 6 to the aptamer-bindable substance 1 and separation of the aptamer 6 from the aptamer-bindable substance 1 occur. As the conditions such as a temperature, a pH, and an electrolyte in the following steps, for example, conditions commonly used in the SELEX method can be applied, and the conditions can be set suitably such that binding of the aptamer 6 to the aptamer-bindable substance 1 and separation of the aptamer 6 from the aptamer-bindable substance 1 occur. However, the electrolyte concentration is preferably set such that the detection accuracy of the electrochemical measurement in the following (1-2) aptamer binding state detection step (A-2) and the following (1-4) aptamer separation state step of detecting (B-2) is not impaired, for example.

(1-1) Probe Provision Step (A)

As the detection method of this Embodiment is performed, first, the aptamer 6 is bound to the aptamer-bindable substance 1 of the probe 4. This probe provision step can be performed any time before the detection of the target substance as long as the effect of the present invention is achieved. The probe provision step can be performed by adding the aptamer 6 to the probe 4, for example (FIGS. 1A and 2A). By adding the aptamer 6 to the probe 4, as the aptamer 6 approaches the probe 4, the aptamer 6 binds to the epitope of the aptamer-bindable substance 1 (FIGS. 1B and 2B).

(1-2) Aptamer Binding State Detection Step (A-2)

In this Embodiment, binding between the aptamer 6 and the aptamer-bindable substance 1 is detected (FIGS. 1C and 2C). This can be achieved by detecting the electron transfer between the electrode reactive substance 2 and the electrode 5, for example. This detection principle can be explained, for example, as follows. That is, by binding the aptamer 6 to the aptamer-bindable substance 1, the mobility of the probe 4 in the solution decreases. Examples of the reason for the decrease in mobility include a decrease in diffusion coefficient, electrostatic interaction between aptamers that are bound to the probe 4, and steric hindrance. The diffusion coefficient decreases as the apparent molecular weight of the probe 4 increases because the aptamer 6 is an anionic polymer, or the like, for example. Therefore, the frequency of contacting the electrode reactive substance 2 of the probe 4 with the electrode 5 through movement of the electrode reactive substance 2 in the solution decreases. Accordingly, in this state, the detection value of the electron transfer between the electrode reactive substance 2 and the electrode 5 is smaller than that before the probe provision step. Thereby, the binding state in which the aptamer 6 is bound to the aptamer-bindable substance 1 can be detected. However, this principle is mere an illustration and does not limit the present invention. The electron transfer between the electrode reactive substance 2 and the electrode 5 can be measured by performing an electrochemical measurement using the electron transfer detection device 13 with the electrode 5 as a working electrode. Specifically, the electrode 5 and the counter electrode 12 are connected to the electrochemical measurement device 13 for performing the electrochemical measurement. Further, for example, in order to perform potential control correctly, the electrochemical measurement may be performed in a three-electrode manner by separately connecting a reference electrode (not shown) to the electrochemical measurement device 13 in addition to the electrode 5 and the counter electrode 12. Here, the aptamer binding state detection step (A-2) may be performed simultaneously with the probe provision step (A) or may be performed after the probe provision step (A).

(1-3) Separation Step (B-1)

After the (1-2) aptamer binding state detection step, a sample is added to the solution. In the case where the sample contains the target substance 8, the target substance 8 approaches the probe 4 that is bound to the aptamer due to the addition (FIG. 1D), and the target substance 8 binds to the aptamer 6 (FIG. 1E). Here, binding between the target substance 8 and the aptamer 6 occurs in the case where the aptamer 6 binds to the target substance 8 more tightly than to the aptamer-bindable substance 1. The aptamer 6 is separated from the aptamer-bindable substance 1 after the aptamer 6 is bound to the target substance 8 (FIG. 1F). In contrast, in the case where the sample does not contain the target substance 8 but contains the non-target substance 9 (FIG. 2D), the non-target substance 9 does not bind to the aptamer 6 (FIG. 2E). Therefore, the aptamer 6 is kept bound to the aptamer-bindable substance 1 (FIG. 2F).

(1-4) Aptamer Separation State Detection Step (B-2)

After the (1-3) separation step, separation of the aptamer from the aptamer-bindable substance 1 that is bound to the aptamer 6 is detected (FIGS. 1G and 2G). This detection can be performed by detecting the probe 4 after separation of the aptamer. That is, in the (1-3) separation step, in the case where the sample contains the target substance 8, when the aptamer 6 is separated from the aptamer-bindable substance 1, the probe 4 is separated from the aptamer 6. Accordingly, for example, it can be determined that the sample contains the target substance 8 if an aptamer-unbound probe that is not bound to the aptamer 6 can be detected. Further, for example, it can be determined that the sample does not contain the target substance 8 if the aptamer-unbound probe cannot be detected. The detection of the aptamer-unbound probe can be performed, for example, by detecting the electron transfer between the electrode reactive substance 2 and the electrode 5.

(1-5) Target Substance Detection Step (B-3)

Then, the target substance is detected by comparing the detection value of the electron transfer detected in the (1-2) aptamer binding state detection step and the detection value of the electron transfer detected in the (1-4) aptamer separation state detection step. This detection principle can be explained, for example, as follows. That is, in the (1-3) separation step, in the case where the sample contains the target substance 8, the probe 4 is separated from the aptamer 6. Since a decrease in mobility of the probe 4 in the solution can thereby be solved, the contact frequency between the electrode reactive substance 2 and the surface of the electrode 5 is recovered, and the detection value of the electron transfer increases (FIG. 1G). In contrast, in the case where the sample does not contain the target substance 8, since the contact efficiency between the electrode reactive substance 2 and the surface of the electrode 5 remains low, the detection value of the electron transfer is not recovered (FIG. 2G). However, this principle is mere an illustration and does not limit the present invention. Here, if the electrode reactive substance 2 is a catalyst (for example, enzyme), the solution preferably contains a reactive substance for the catalyst (for example, a substrate of enzyme), and the detection principle of this case can be explained, for example, as follows. That is, when the aptamer 6 is bound to the aptamer-bindable substance 1, the mobility of the probe 4 in the solution decreases. Therefore, the frequency of contacting the catalyst 2 contained in the probe 4 with the electrode 5 through the movement of the catalyst 2 in the solution decreases. Since the reactive substance (substrate) is added to the reaction solution, when the catalyst 2 that has transferred electrons with the reactive substance (substrate) is brought into contact with the electrode 5, the electron transfer between the catalyst 2 and electrode 5 occurs. That is, in the state where the contact frequency between the catalyst 2 and the electrode 5 is low, the detection value of the electron transfer between the catalyst 2 and the electrode 5 is smaller than that before the probe provision step. Thereby, the binding state in which the aptamer 6 is bound to the aptamer-bindable substance 1 can be detected. Here, an electron transfer mediator may be added to the solution. In this case, the electron transfer occurs when the catalyst (enzyme) that has transferred electrons with the reactive substance (substrate) transfers electrons with the electron transfer mediator and the electron transfer mediator electrochemically reacts with the electrode. Specifically, as the contact frequency between the electrode and the catalyst increases, the number of redox cyclings of the electron transfer mediator increases and the frequency of an electrode reaction thereby increases. Therefore, an increase in the detection value of the electron transfer can be detected. More than one electrode reaction is generated due to the change of the mobility of the electrode reactive substance per molecule by the redox cycling. As a result, for example, the signal is amplified and the S/N ratio can further be increased. Further, the electron transfer mediator may be immobilized to the electrode, for example. In this case, as the contact frequency between the electrode and the catalyst increases, the contact frequency between the electron transfer mediator and the catalyst increases and the number of redox cyclings thereby increases. Thereby, the increase in the detection value of the electron transfer can be detected. However, the principle as described above is mere an illustration and does not limit the present invention. In this Embodiment, by detecting increase or decrease of the probe 4 that is bound to the aptamer 6, separation of the aptamer 6 that is bound to the probe 4 from the aptamer-bindable substance 1 is detected. However, detection of the target substance 8 is not limited to the Embodiment described above as long as the separation of the aptamer 6 from the aptamer-bindable substance 1 that is bound to the aptamer 6 can be detected.

According to the method of detecting a target substance of this Embodiment, the detection value of the electron transfer between the electrode reactive substance 2 and the electrode 5 increases or decreases depending on the case in which the aptamer 6 is bound to the aptamer-bindable substance 1 in the probe 4 or the case in which the aptamer 6 is not bound to the aptamer-bindable substance 1 in the probe 4. Specifically, according to the detection method of this Embodiment, the detection value of the electron transfer between the electrode reactive substance 2 and the electrode 5 increases in the case where the aptamer 6 is bound to the aptamer-bindable substance 1 and decreases in the case where the aptamer 6 is not bound to the aptamer-bindable substance 1, for example. By utilizing this, it can be checked whether the sample contains the target substance 8. According to this Embodiment, since the probe 4 includes the electrode reactive substance 2, there is no need to modify the aptamer 6 with the electrode reactive substance 2. Further, since the aptamer 6 is immobilized to the electrode 5 by the probe 4, there is no need to modify the aptamer 6 with a functional group for formation of a crosslinking reaction with an electrode surface. In this manner, the detection method of this Embodiment can be performed very simply. Further, this Embodiment can detect the target substance without depending on the conformational change of the aptamer, for example. Therefore, this Embodiment can be applied to various aptamers and target substances and has high general versatility. Further, in the detection method of this Embodiment, for example, the detection value of the electron transfer increases as the concentration of the target substance increases. Therefore, the detection method of this Embodiment shows a signal-increasing type detection reaction and achieves a high S/N ratio.

In this Embodiment, as described above, at the time of the (1-1) probe provision step (A), the probe 4 is preliminarily immobilized to the electrode 5. However, this Embodiment is not limited thereto. For example, the probe 4 may be immobilized to the electrode 5 before or after the aptamer is bound to the aptamer-bindable substance in the probe provision step (A). Thereby, a treatment of applying a reaction solution to the electrode 5, a treatment of immersing the electrode 5 in a reaction solution, or the like can be omitted, and a detection operation can be simplified, for example. Further, for example, in the probe provision step (A), immediately after the aptamer 6 is added to the aptamer-bindable substance 1, for example, a step of binding the aptamer 6 to the aptamer-bindable substance 1 by bringing the mixture of the probe 4 and the aptamer 6 into contact with the electrode surface and a step of immobilizing the probe 4 to the electrode 5 may be performed at the same time. Thereby, the detection operation can be simplified, for example.

In this Embodiment, a known detection value can be used as a measurement value of the electron transfer efficiency in the (1-2) binding state detection step. Thereby, for example, the (1-2) aptamer binding state detection step can be omitted. The known detection value can be calculated, for example, from the density of the probe on the electrode and the ratio between the number of probes that are bound to the aptamers and the number of probes that are not bound to the aptamers at the time of the (1-1) probe provision step (A). That is, for example, on the basis of the detection value of the electron transfer, the detection value of the electron transfer between the electrode reactive substance and the electrode can preliminarily be obtained using an electrode to which the probe 4 and the aptamer 6 are bound under known conditions. Then, the target substance can be detected from the difference between the known detection value and the detection value of the electron transfer detected in the (1-4) aptamer separation state detection step (B-2). As a result, the detection operation can further be simplified according to this Embodiment.

This Embodiment may further include an unbound aptamer removing step of removing the aptamer that is nonspecifically adsorbed to the electrode surface and is not bound to the probe prior to the (1-3) separation step (B-1), for example. Thereby, for example, in the (1-3) separation step (B-1), the target substance 8 can be prevented from binding to an aptamer that is nonspecifically adsorbed to the electrode surface. As a result, for example, the reproducibility of the amount of the aptamer that is separated from the probe that is bound to the aptamer can be improved, and a detection result that reflects the presence of the target substance more correctly can be obtained.

This Embodiment can be performed using the aptamers that are obtained in Embodiments 3 to 7 that will be described below, for example. Since the aptamers of Embodiments 3 to 7 that will be described below are recovered using the same probe as this Embodiment, for example, they show a particularly high degree of selectivity when they are used in this Embodiment.

### Embodiment 2

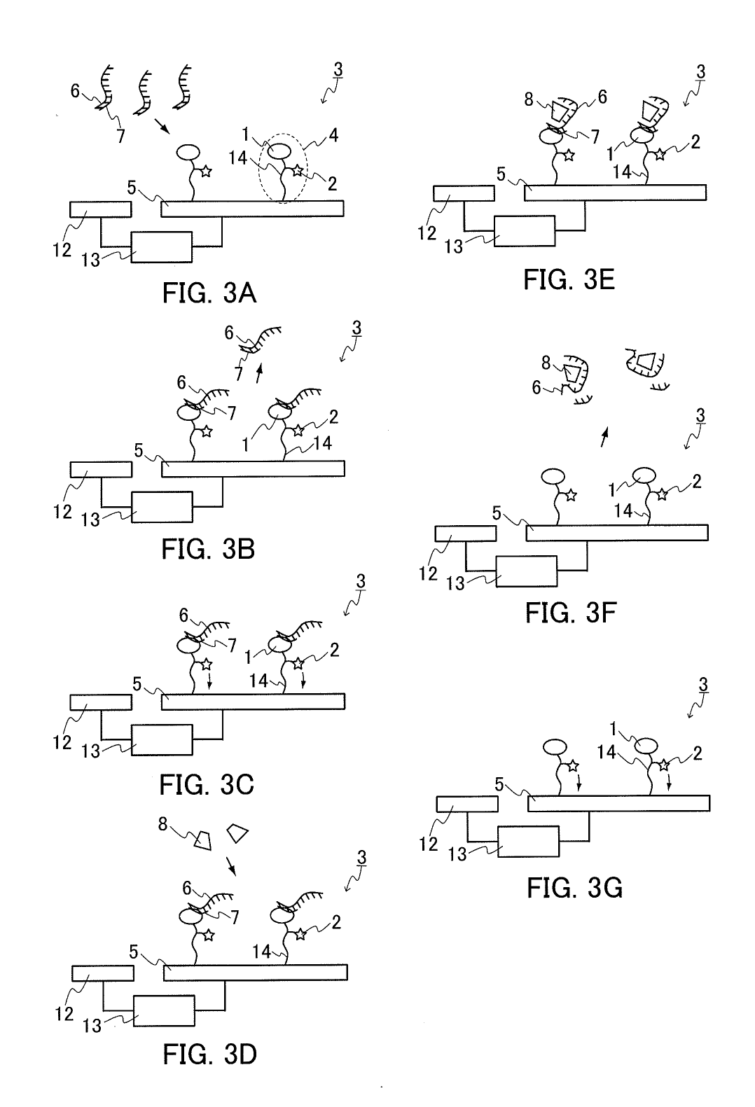

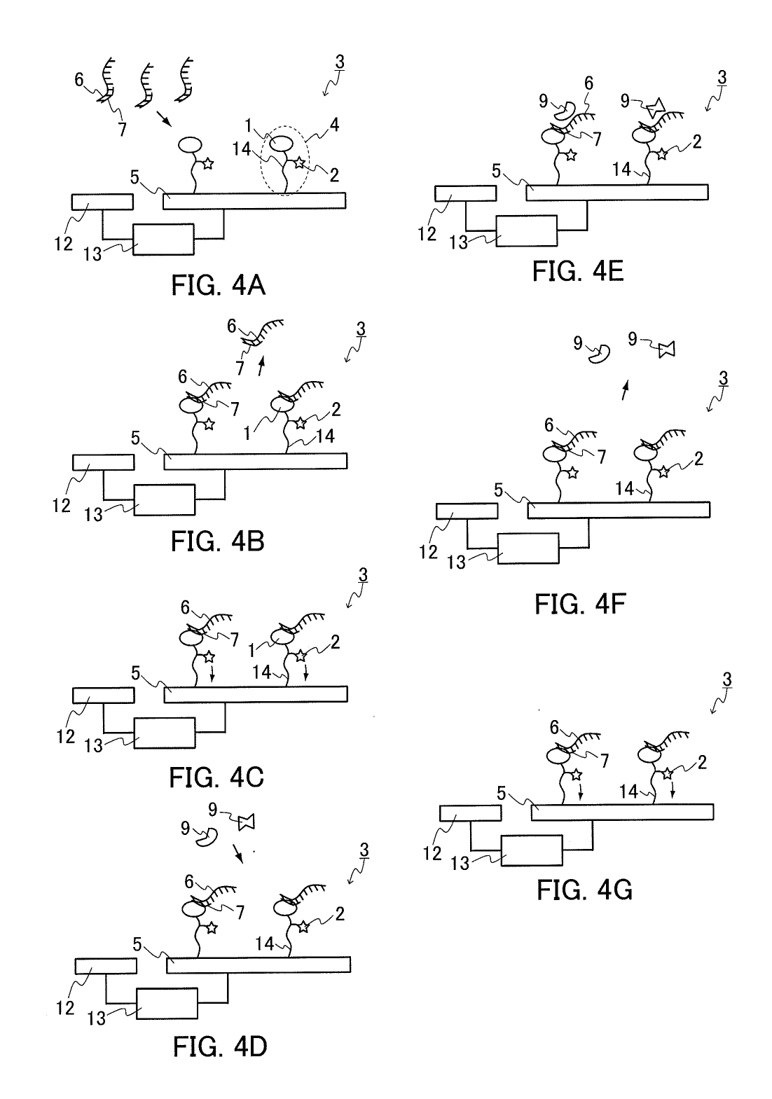

This Embodiment shows another example of the probe, the method of detecting a target substance, and the target substance detection apparatus of the present invention. The detection method of this Embodiment is the same as that of Embodiment 1 except that an aptamer having a double-stranded nucleic acid part is used as the aptamer and a probe including an aptamer-bindable substance that binds to the double-stranded nucleic acid part of the aptamer is used as the probe. FIG. 3 shows an example in which a target substance 8 is added to the solution that will be described below. FIG. 4 shows an example that is similar to the example shown in FIG. 3 except that a non-target substance 9 is added instead of the target substance 8. The detection method of this Embodiment can be performed using the probe and the target substance detection apparatus of this Embodiment shown in FIGS. 3 and 4, for example. In this Embodiment, an aptamer-bindable substance 1 is, for example, an intercalator, a nucleic acid binding protein, or the like, and the aptamer-bindable substance 1 itself can be an electrode reactive substance. An aptamer 6 has a double-stranded nucleic acid part in which a part of the nucleic acid is hybridized (FIGS. 3A and 4A). Such an aptamer can be produced by hybridizing a part of the single-stranded nucleic acid 6, which is an aptamer, to a single-stranded nucleic acid fragment 7 having a base sequence complementary thereto, for example. Also, such an aptamer 6 can be produced by hybridizing base sequences that are complementary to each other of a single-stranded nucleic acid, which is an aptamer, to form a double-stranded nucleic acid part, for example. In the probe 4, the aptamer-bindable substance 1 has a property of specifically binding to a double-stranded part of the nucleic acid 6 but not binding to a single-stranded part of the nucleic acid 6. The following steps of this Embodiment are performed in a solution, for example. The temperature, composition, electrolyte, pH, and the like of the solution can be set as with Embodiment 1. However, preferably, the conditions are preferably set such that binding of a double-stranded part of the aptamer 6 can be maintained and the aptamer-bindable substance 1 can be bound to the double-stranded part of the aptamer 6 in the following (2-1) step.

(2-1) Probe Provision Step (A)

As the detection method of this Embodiment is performed, first, the aptamer 6 is bound to the aptamer-bindable substance 1 of the probe 4. This probe provision step can be performed any time before the detection of the target substance 8 as long as the effect of the present invention is achieved. The probe provision step can be performed by adding the aptamer 6 to the probe 4, for example. By adding the aptamer 6 to the probe 4, as the aptamer 6 approaches the probe 4, the aptamer-bindable substance 1, which is a double-stranded nucleic acid aptamer-bindable substance, binds to the double-stranded nucleic acid part of the aptamer 6 (FIGS. 3B and 4B). Thereby, the aptamer 6 binds to the probe 4 via the aptamer-bindable substance 1.

(2-2) Aptamer Binding State Detection Step (A-2)

Next, binding between the aptamer 6 and the aptamer-bindable substance 1 is detected (FIGS. 3C and 4C). This binding can be detected by measuring the electron transfer between the electrode reactive substance 2 and the electrode 5 using an electron transfer detection device 13, for example. Here, the aptamer binding state detection step (A-2) may be performed simultaneously with the probe provision step (A) or may be performed after the probe provision step (A).

(2-3) Separation Step (B-1)

After the (2-2) aptamer binding state detection step, a sample to be detected is added to the solution in the same manner as in Embodiment 1. In the case where the sample contains the target substance 8, due to the addition, as the target substance 8 approaches the aptamer 6 that is bound to the aptamer-bindable substance 1 (FIG. 3D), the target substance 8 binds to the aptamer 6 (FIG. 3E). Here, when the target substance 8 binds to the aptamer 6, a branch migration is caused and the double-stranded part of the aptamer 6 is lost. The branch migration is caused in the case where the binding between the target substance 8 and the aptamer 6 is stronger than the binding between nucleic acids that form the double-stranded part of the aptamer 6. In this manner, when the double-stranded part of the aptamer 6 is lost, the aptamer-bindable substance 1 cannot maintain the binding with the aptamer 6, and is separated from the aptamer 6 (FIG. 3F). In contrast, in the case where the sample does not contain the target substance 8 but contains the non-target substance 9 (FIG. 4D), the non-target substance 9 does not bind to the aptamer 6 (FIG. 4E). Therefore, the aptamer 6 is kept bound to the aptamer-bindable substance 1 (FIG. 4F).

(2-4) Aptamer Separation State Detection Step (B-2)

After the separation step, separation of the aptamer 6 from the aptamer-bindable substance 1 is detected (FIGS. 3G and 4G). This detection can be performed using the electrochemical measurement device 13 in the same manner as in Embodiment 1, for example.

(2-5) Target Substance Detection Step (B-3)

In the same manner as in Embodiment 1, the target substance is detected by comparing the electron transfer measured in the (2-2) aptamer binding state detection step and the electron transfer measured in the (2-4) aptamer separation state detection step.

According to the detection method of this Embodiment, for example, the target substance can be detected specifically without immobilizing a substance having an epitope that is identical to or similar to the whole or a part of the target substance 8 to an electrode. Accordingly, for example, the detection method of this Embodiment can be applied to various aptamers each having a double-stranded part in its sequence, and has high general versatility. Further, with respect to the detection method and the target substance detection apparatus using the aptamer of this Embodiment, for example, the same probe can be applied to various target substances. Therefore, general versatility is high and the production process can be simplified.

This Embodiment can be performed using the aptamers obtained in the following Embodiments 3 to 7, for example. Since the aptamer of the following Embodiment 7 is recovered using the same probe as this Embodiment under the same conditions as this Embodiment, the aptamer is suitable for the detection method of this Embodiment, and a high degree of selectivity can be achieved with a simple operation.

### Embodiment 3

This Embodiment shows an example of the screening method and the aptamer screening apparatus of the present invention. The screening method of this Embodiment can be performed using the aptamer screening apparatus of this Embodiment shown in FIG. 1A. The aptamer screening apparatus of this Embodiment further includes a recovering unit in addition to the target substance detection apparatus of the present invention of Embodiment 1. In other words, the aptamer screening apparatus of this Embodiment includes a probe 4 in which an aptamer-bindable substance 1 and a labeling substance 2 are each bound to a linker 14, a recovering unit, and a support 5 that immobilizes the probe 4. In this Embodiment, the recovering unit is a buffer solution. An aptamer 6 may be a nucleic acid or a peptide.

The screening method of this Embodiment includes the following steps (A′) to (C′). (A′) a first step of supplying the aptamer candidate substance 6 to the probe 4 to which an aptamer is specifically bindable.

(B′) a second step of separating the aptamer candidate substance 6 from the probe 4 by supplying a target substance 8 to the probe 4 and binding the aptamer candidate substance 6 that is bound to the probe 4 to the target substance 8.
(C′) a third step of recovering the aptamer candidate substance 6 separated.

FIG. 5 shows the mechanism of the screening method of this Embodiment. This Embodiment describes an example in which an aptamer is recovered using the same probe as Embodiment 1. That is, in this Embodiment, the probe 4 specifically binds to the aptamer 6 by the aptamer-bindable substance 1. In this Embodiment, the following steps are performed in a solution, for example. The composition of the solution is the same as that of Embodiment 1. In this Embodiment, the aptamer candidate substance may be a nucleic acid or a peptide.

(3-1) First Step (A′)

First, the aptamer candidate substance 6 is added to the probe 4. In the case where the aptamer candidate substance 6 has the structure of specifically binding to the aptamer-bindable substance 1, the aptamer candidate substance 6 binds to the aptamer-bindable substance 1. In this Embodiment, since the aptamer-bindable substance 1 is immobilized to the electrode 5 via a linker 14, the aptamer candidate substance 6 is immobilized to the electrode 5 via the probe 4 (FIG. 5A). In the case where the aptamer candidate substance 6 does not have the structure of binding to the aptamer-bindable substance 1, the aptamer candidate substance 6 does not bind to the aptamer-bindable substance 1 (FIG. 5A). The electrode 5 is used as the support in this Embodiment. However, this Embodiment is not limited thereto and any support is applicable as long as it can be used as the support.

(3-2) Second Step (B′)

After the first step, a sample containing the target substance 8 is added to the solution. When the target substance 8 approaches the probe to which the aptamer candidate substance 6 is bound, in the case where the aptamer candidate substance 6 has the structure of the aptamer of the target substance 8, the aptamer candidate substance 6 binds to the target substance 8 (FIG. 5B). In the case where the aptamer candidate substance 6 does not have the structure of the aptamer of the target substance 8, the aptamer candidate substance 6 does not bind to the target substance 8 (FIG. 5B). In the case where the aptamer candidate substance 6 is bound to the target substance 8, following that, the aptamer candidate substance 6 is separated from the aptamer-bindable substance 1 that is bound to the aptamer candidate substance (FIG. 5C). Here, the separation is caused in the case where the binding between the target substance 8 and the aptamer candidate substance 6 is stronger than the binding between the aptamer-bindable substance 1 and the aptamer candidate substance 6.

(3-3) Third Step (C′)

After the second step, the aptamer candidate substance 6 that is bound to the target substance 8 and separated from the aptamer-bindable substance 1 is recovered. The aptamer candidate substance 6 having the structure of the aptamer of the target substance 8 is in the state in which it is distanced away from the electrode 5 because it is separated from the aptamer-bindable substance 1. Therefore, for example, by pouring a solution such as a buffer onto the surface of the electrode 5, the aptamer candidate substance 6 having the structure of the aptamer of the target substance 8 can be recovered. Since the aptamer candidate substance that does not have the structure of the aptamer of the target substance 8 is kept bound to the aptamer-bindable substance 1 (FIG. 5C), the aptamer candidate substance keeps the state in which it is immobilized to the electrode 5. Therefore, in the third step, only the aptamer candidate substance 6 that has the structure of the target substance 8 can be recovered.

According to the screening method of this Embodiment, the aptamer candidate substance 6 is kept bound to the probe 4 in the case where the target substance 8 is not present, and the aptamer candidate substance 6 binds to the target substance 8 and is separated from the aptamer-bindable substance 1 in the case where the target substance 8 is present. Therefore, in the latter case, the aptamer candidate substance 6 can be obtained. Such an aptamer candidate substance 6 is very suitable for the method of detecting a target substance for detecting the target substance 8, for example.

In the screening method of this Embodiment, many types of the aptamer candidate substances are preferably provided. Use of various aptamer candidate substances makes it possible to select the aptamer candidate substance that has a higher binding ability to the target substance, for example. According to the screening method of this Embodiment, for example, a step of modifying the aptamer candidate substance with the electrode reactive substance, functional group, or the like is unnecessary, and the aptamer candidate substance can be recovered efficiently even in the case where various aptamer candidate substances are used.

Preferably, this Embodiment further includes a fifth step of removing the aptamer candidate substance 6 that is not bound to the aptamer-bindable substance 1 prior to the (3-2) second step (B′), for example. Thereby, for example, the one that is not the aptamer candidate substance of the target substance 8 in the third step (C′) can be prevented from being mixed in the aptamer candidate substance recovered in the third step, and the performance of recovery of aptamer can be increased. This Embodiment may further include a sixth step of removing the aptamer candidate substance that is not bound to the target substance in the (3-3) third step (C′), for example.

This Embodiment may further include an analog binding aptamer candidate substance removing step of removing the aptamer candidate substance that is bound to the target substance analog after the aptamer candidate substance that is bound to the aptamer-bindable substance 1 is brought into contact with a target substance analog having a structure similar to the target substance prior to the (3-2) second step, for example. Thereby, for example, the aptamer candidate substance 6 that is bound to the analog can be removed, and the aptamer candidate substance having higher specificity to the target substance 8 can be recovered.

The aptamer obtained by the screening method of this Embodiment can be bound to both of the target substance and the aptamer-bindable substance, for example, and the aptamer binds to the target substance more tightly than to the aptamer-bindable substance. Accordingly, the aptamer can be applied suitably to detection methods using aptamers, for example. Since the aptamer recovered in this Embodiment can be obtained using the same probe as Embodiment 1 under the same conditions as Embodiment 1, for example, it is very suitable for the sensor of Embodiment 1.

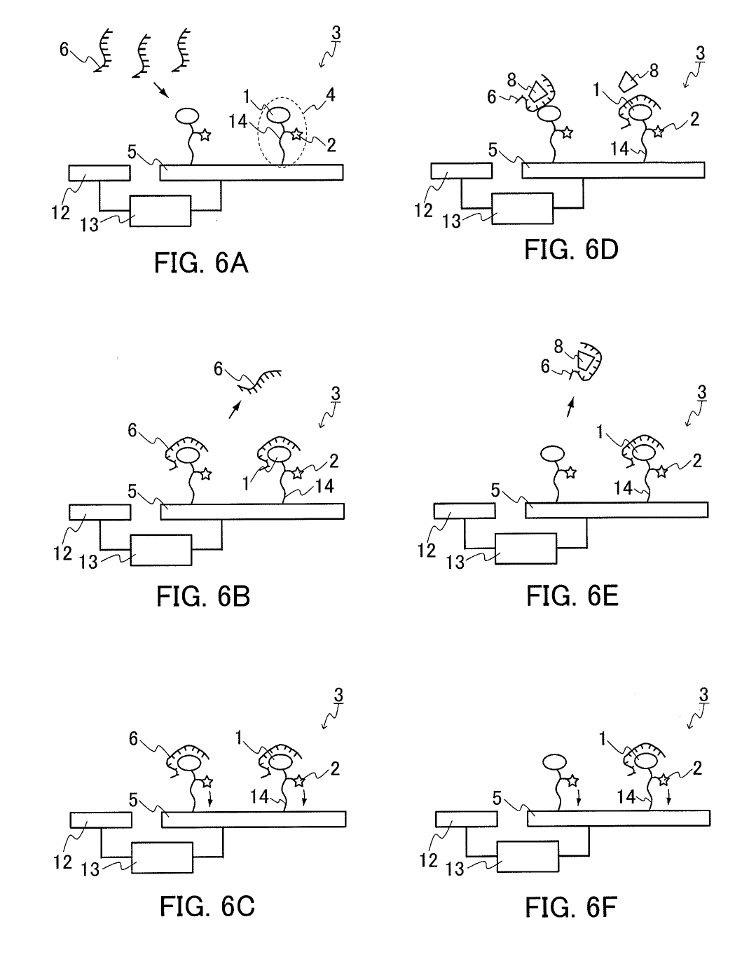

In this Embodiment, prior to the (3-2) second step, the probe may be immobilized to the electrode, for example. That is, as shown in FIG. 6, the probe 4 may be immobilized to the electrode 5 simultaneously with the (3-1) first step of binding the aptamer candidate substance 6 or the probe 4 may be immobilized to the electrode 5 after the (3-1) first step. Thereby, the separation status of the aptamer candidate substance in each of the aforementioned steps can be monitored, for example. Further, for example, a step of applying a reaction solution to the electrode 5, a step of washing the electrode 5, or the like can be omitted suitably, and the steps described above can be simplified.

According to the aptamer screening apparatus of this Embodiment, for example, the aptamer candidate substance capable of detecting a target substance can be recovered without modifying the aptamer candidate substance with an electrode reactive substance, a functional group, or the like, and the aptamer candidate substance can be recovered efficiently even in the case where various aptamer candidate substances are used.

### Embodiment 4

This Embodiment shows another example of the screening method and the aptamer screening apparatus of the present invention. This Embodiment is the same as Embodiment 3 except that the electron transfer between the electrode reactive substance and the electrode is detected during an aptamer recovery operation. That is, in this Embodiment, an aptamer is recovered using the aptamer screening apparatus 3 shown in FIG. 6. The configuration of the aptamer screening apparatus 3 of this Example is the same as that of the target substance detection apparatus 3 of Embodiment 1 except that it includes a recovering unit (not shown). Further, the following steps of this Embodiment are performed in the same solution as Embodiment 3. FIG. 6 shows the mechanism of the screening method of this Embodiment. The aptamer candidate substance 6 may be a nucleic acid or a peptide.

(4-1) First Step (A′)

First, the same treatment as the (3-1) first step of Embodiment 3 is performed to bind an aptamer candidate substance 6 to an aptamer-bindable substance 1. However, this Embodiment is different from Embodiment 3 in that an electrode 5 and a counter electrode 12 are connected to an electrochemical measurement device 13 (FIG. 6A). In this Embodiment, following that, the electron transfer between the electrode reactive substance 2 and the electrode 5 is detected. The electron transfer can be detected in the same manner as in Embodiment 1 by using the electron transfer detection device 13, for example. Here, since the aptamer candidate substance 6 is bound to the aptamer-bindable substance 1, the mobility of the probe 4 in a solution is lower than that before the first step. Therefore, the detection value of the electron transfer between the electrode reactive substance 2 and the electrode 5 is lower than that before the first step (FIG. 6C).

(4-2) Second Step (B′)

Next, the same treatment as the (3-2) second step of Embodiment 3 is performed. Thereby, as described in Embodiment 3, the aptamer candidate substance 6 binds to the target substance 8 (FIG. 6D). Further, as described in Embodiment 3, the aptamer candidate substance 6 that is bound to the target substance 8 is separated from the aptamer-bindable substance 1.

(4-3) Third Step (C′)

Next, the same treatment as the (3-3) third step of Embodiment 3 is performed. Through this step, the aptamer candidate substance 6 that is separated from the aptamer-bindable substance 1 can be recovered. However, in this Embodiment, the aptamer candidate substance 6 recovered in the third step is detected. By the (4-2) second step, the aptamer candidate substance 6 having the structure of the aptamer of the target substance 8 is in the state where it is separated from the probe 4. In this manner, by separating the aptamer candidate substance 6 from the probe 4, the mobility of the probe 4 in a solution increases and the detection value of the electron transfer between the electrode reactive substance 2 and the electrode 5 increases (FIG. 6F). That is, when the electron transfer between the electrode reactive substance 2 and the electrode 5 is detected, for example, by the electrochemical measurement device 13 and the change from the detection value of the electron transfer detected in the (4-1) first step is checked, the aptamer candidate substance 6 separated can be detected. Since the aptamer candidate substance 6 separated in the (4-2) second step is recovered in the (4-3) third step, the aptamer candidate substance 6 recovered in the third step can be detected through the detection of the electron transfer.

By the use of the screening method of this Embodiment, it can be evaluated immediately whether the aptamer candidate substance 6 is recovered efficiently with the change of amount of the electron transfer between the electrode reactive substance 2 and the electrode 5 as an indicator, for example. Specifically, the more the binding strength between the aptamer candidate substance 6 and the target substance, the more the increase in the detection value of the electron transfer because many aptamer candidate substances are separated from the aptamer-bindable substances 1. According to the screening method of this Embodiment, the recovery efficiency and productivity of the aptamer candidate substance are increased.

According to the screening method of this Embodiment, it can be detected immediately whether the aptamer candidate substance of the target substance is separated, for example, and it can be evaluated simply whether the conditions such as composition, a pH, and a temperature of the solution used in each of the steps for recovering the aptamer candidate substance are appropriate. Therefore, the efficiency and productivity of recovery operation of aptamer can be increased.

### Embodiment 5

This Embodiment shows another example of the screening method and the aptamer screening apparatus of the present invention. This Embodiment is the same as Embodiment 4 except that the electron transfer between the electrode reactive substance and the electrode is detected before an aptamer candidate substance recovery operation. That is, in this Embodiment, the same aptamer screening apparatus of the present invention as that used in Embodiment 4 is used. Also in this Embodiment, the following steps are performed in a solution. The composition of the solution is not particularly limited as long as the binding reaction between the aptamer-bindable substance and the aptamer occurs.

In this Embodiment, the procedure from the (4-1) first step to the (4-3) third step in Embodiment 4 is performed. However, in this Embodiment, the electron transfer between the electrode reactive substance 2 and the electrode 5 can be detected before the aptamer candidate substance is recovered in the (4-3) third step, for example. For example, this measurement can be performed between the (4-2) second step and the (4-3) third step, right before the (4-3) third step, or the like, and the number of measurements is not limited. The electron transfer can be detected in the same manner as in Embodiment 1 by using the electron transfer detection device 13. As described in Embodiment 4, the aptamer candidate substance 6 can be detected by detecting the electron transfer. In this Embodiment, the electron transfer can be detected continuously in each of the aforementioned steps. For example, by detecting the aptamer candidate substance separated from the aptamer-bindable substance before the aptamer candidate substance is recovered in the third step, for example, the process of separating the aptamer candidate substance 6 from the aptamer-bindable substance 1 that is bound to the aptamer candidate substance can be monitored. Thereby, by setting the amount of separation of the intended aptamer, an appropriate timing such as the termination point of the (4-1) first step, the (4-2) second step, or the like or the starting point of the (4-3) third step can be decided with the detection value of the electron transfer as an indicator. Further, for obtaining an intended amount of aptamer, for example, the conditions such as composition, a pH, and a temperature of the solution can be changed during the (4-2) second step. Thereby, the efficiency of the recovery operation of aptamer can be increased. Further, in this Embodiment, the detection of the electron transfer that is performed at the time when the aptamer candidate substance is recovered in the (4-3) third step may be performed or not performed, for example. For example, in the case where the termination point of the separation of the aptamer candidate substance from the aptamer-bindable substance can be determined by the measurement that is performed right before the (4-3) third step, the detection of the electron transfer can be omitted.

According to this Embodiment, for example, the termination point of each of the aforementioned steps can be determined and the conditions for separating the aptamer candidate substance from the aptamer-bindable substance can be optimized with the change of the detection value of the electron transfer as an indicator. Thereby, the recovery efficiency of the aptamer candidate substance is increased.

### Embodiment 6

This Embodiment shows another example of the screening method and the aptamer screening apparatus of the present invention. This Embodiment is the same as Embodiment 4 except that the screening method further includes a fourth step (D′) of amplifying the aptamer candidate substance that has been recovered in the third step (C′) of Embodiment 4, and the procedure from the first step (A′) to the third step (C′) or the procedure from the first step (A′) to the fourth step (D′) is performed repeatedly using the aptamer candidate substance amplified in the fourth step (D′). That is, in this Embodiment, the same aptamer screening apparatus of the present invention as that used in Embodiment 4 and an aptamer amplifying unit (not shown) are used. Here, this Embodiment will be described with reference to the case in which the aptamer candidate substance is a nucleic acid as an example. The following steps are performed in a solution, for example. The conditions such as composition and the like of the solution are the same as those in Embodiment 4.

In this Embodiment, the same treatments as those from the (4-1) first step to the (4-3) third step of Embodiment 4 are performed. In this Embodiment, after the (4-3) third step, the following (6-1) fourth step is performed.

(6-1) Fourth Step

In this Embodiment, for example, the aptamer candidate nucleic acid recovered in the (4-3) third step is amplified by a nucleic acid amplification method such as polymerase chain reaction (PCR). In the case of using PCR, preferably, a primer sequence for amplification is preliminarily applied to the aptamer candidate nucleic acid, for example. In this Embodiment, following that, the (4-1) first step is performed. That is, the aptamer candidate nucleic acid amplified is used in another first step, and the procedure after the (4-1) first step is performed. In this Embodiment, the procedure from the (4-1) first step to the (4-3) third step or the procedure from the first step (4-1) to the fourth step (6-1) can be repeated in this manner. The aptamer candidate nucleic acid recovered through a series of an aptamer candidate nucleic acid recovery operation from the (4-1) to the (4-3) may be a mixture of aptamers having different structures. However, by repeating the steps for recovering the aptamer as in this Embodiment, the aptamer candidate nucleic acid having a high binding strength with the target substance can be enriched, for example, and an aptamer more suitable for detection of a target substance can be obtained.

In this Embodiment, in the (4-1) first step, for example, a mixture (nucleic acid pool) of nucleic acids having various sequences may be added to the aptamer candidate nucleic acid if necessary. Thereby, for example, an aptamer having a higher target substance binding ability can be selected from various aptamer candidate nucleic acids. The mixture of nucleic acids can be added to the aptamer candidate nucleic acid by amplifying a nucleic acid by a method in which an error occurs in the (6-1) fourth step or by adding a nucleic acid mixture synthesized separately to the nucleic acid that has been amplified in the (6-1) fourth step, for example.

In this Embodiment, for example, before the completion of the aptamer recovery operation, the electron transfer between the electrode reactive substance and the electrode may be detected at least once. Thereby, for example, the separation state of the aptamer candidate nucleic acid can be monitored in each of the aforementioned steps. Therefore, for example, after checking the separation of the intended aptamer candidate nucleic acid, the termination point of the repeat of the aptamer recovery operation started from the (4-1) first step can be decided. Further, at the time of repeating the steps for recovering the aptamer, for example, the conditions such as composition, a pH, and a temperature of the solution used in each of the aforementioned steps can be decided on the basis of the separation status of the aptamer candidate nucleic acid monitored. Therefore, according to this Embodiment, the efficiency of the aptamer recovery operation is increased.

### Embodiment 7

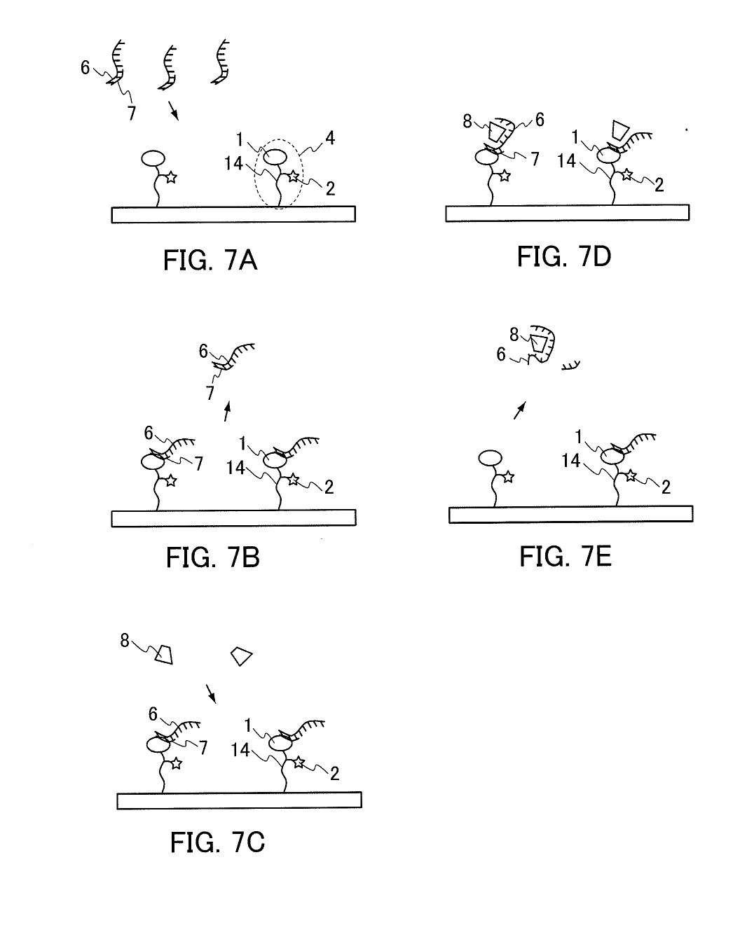

This Embodiment shows another example of the screening method and the aptamer screening apparatus of the present invention. This Embodiment is the same as Embodiment 4 except that the aptamer and the probe used in Embodiment 2 are used. The configuration of the aptamer screening apparatus of this Example is the same as that of the target substance detection apparatus 3 of Embodiment 2 except that it includes a recovering unit (not shown). The conditions such as a temperature, a pH, and an electrolyte in the following steps are the same as those in Embodiment 2. FIG. 7 shows the mechanism of the screening method of this Embodiment.

(7-1) First Step (A′)

First, the aptamer candidate nucleic acid 6 is added to the probe 4 (FIG. 7A). In the case where the aptamer candidate nucleic acid 6 has a double-stranded nucleic acid part, the aptamer candidate nucleic acid 6 binds to the aptamer-bindable substance 1 at the double-stranded nucleic acid part (FIG. 7B). Thereby, the aptamer candidate nucleic acid 6 is immobilized to the electrode 5 via the probe 4 (FIG. 7B). The electrode 5 is used as the support in this Embodiment. However, this Embodiment is not limited thereto and any support is applicable as long as it can be used as the support.

(7-2) Second Step (B′)

After the first step, a sample containing a target substance 8 of an aptamer candidate nucleic acid is added to the reaction solution (FIG. 7C). When the target substance 8 approaches the aptamer-bindable substance 1 that is bound to the aptamer candidate nucleic acid 6, in the case where the aptamer candidate nucleic acid 6 has the sequence of the aptamer of the target substance 8, the aptamer candidate nucleic acid 6 binds to the target substance 8 (FIG. 7D). In the case where the aptamer candidate nucleic acid 6 does not have the sequence of the aptamer of the target substance 8, the aptamer candidate nucleic acid 6 does not bind to the target substance 8 (FIG. 7D). When the aptamer candidate nucleic acid 6 binds to the target substance 8, the double-stranded nucleic acid part is separated (FIG. 7E). Thereby, the aptamer candidate nucleic acid 6 is separated from the aptamer-bindable substance 1 and is also separated from the probe (FIG. 7E). In contrast, in the case where the aptamer candidate nucleic acid 6 does not have the sequence of the target substance 8, since the double-stranded nucleic acid part is not separated, the aptamer candidate nucleic acid 6 is kept bound to the probe (FIG. 7E).

(7-3) Third Step (C′)

After the second step, the aptamer candidate nucleic acid 6 that is separated from the probe 4 bound to the aptamer candidate nucleic acid 6 is recovered. The recovery can be performed using the same method as Embodiment 4.

According to this Embodiment, for example, the aptamer that is bound to the target substance can be recovered without immobilizing a molecule having an epitope that is identical to or similar to the whole or a part of the target substance to the electrode. This Embodiment can be applied to various aptamer candidate nucleic acids each having a double-stranded part in its sequence, and has high general versatility. For example, even with respect to a substance that does not have a functional group that can be used for a reaction for immobilizing to a substrate, a substance having a high degradability, and a substance whose aptamer could hardly be obtained because the epitope could not be immobilized to the substrate, by bringing the solutions thereof into contact with the aptamer candidate nucleic acid, the aptamer can be obtained. Further, with respect to the substance in which the epitope has conventionally been vanished or the epitope has conventionally been hidden due to the immobilization, the aptamer can be obtained.

Preferably, this Embodiment also includes a fifth step of removing the aptamer candidate substance that is not bound to the aptamer-bindable substance prior to the (7-2) second step, for example. Thereby, for example, the one that is not the aptamer candidate nucleic acid of the target substance 8 can be prevented from being mixed in the aptamer recovered in the third step, and the purity of the aptamer in the target substance in the recovered substance can be increased, and therefore the recovery of the aptamer can be performed efficiently. Further, this Embodiment may further include a sixth step of removing the aptamer candidate substance that is not bound to the target substance in the (7-3) third step (C′), for example.

Further, in this Embodiment, from the (4-1) first step to the completion of the aptamer recovery operation of this Embodiment, the electron transfer between the electrode reactive substance 2 and the electrode 5 may be detected at least once, for example. Thereby, for example, the separation state of the aptamer candidate nucleic acid 6 can be monitored. Further, the obtainment status of the aptamer can be monitored with the detected detection value of the electron transfer as an indicator, and the efficiency of the aptamer recovery operation can be increased.

Further, in this Embodiment, similar to Embodiment 6, the aptamer candidate nucleic acid recovered in the (4-3) third step may be amplified, and the aptamer recovery operation started from the (4-1) first step may be repeated using the aptamer candidate nucleic acid amplified. Here, in the case where the aptamer candidate nucleic acid is amplified, for example, by PCR, a primer sequence may be applied only to the sequence of the aptamer candidate nucleic acid. From this, since a single-stranded nucleic acid fragment recovered together with the aptamer candidate nucleic acid is not amplified, the performance of recovering aptamer is not impaired. By repeating the steps for recovering the aptamer, a higher-performance aptamer can be recovered. Since this Embodiment can be performed using the same probe as the detection method of the present invention under the same conditions as the detection method of the present invention, for example, an aptamer that is very suitable for the detection method of the present invention can be obtained.

Since the aptamer recovered in this Embodiment is recovered especially using the same probe as Embodiment 2 under the same conditions as Embodiment 2, in the case where it is used in Embodiment 2, it shows a particularly high degree of selectivity. In this Embodiment, since the same probe can be applied to various target substances, general versatility can be increased, and the recovery of the aptamer can be performed efficiently.

### EXAMPLES

Hereinafter, Examples of the present invention will be described. However, the present invention is not limited to these Examples.

### Example 1

In this Example, using the probe 4 and the target substance detection apparatus 3 of the present invention illustrated in FIG. 1A, thrombin was detected by the detection method of the present invention with the aptamer that is specifically bound to thrombin. As shown in FIG. 1A, the target substance detection apparatus 3 of the present invention used in this Example includes a probe 4 in which an aptamer-bindable substance 1 that is specifically bound to an aptamer 6 and an electrode reactive substance 2 are each bound to an end of a linker 14; an electrode 5; and an electron transfer detecting unit 13. In this Example, the aptamer 6 is an aptamer that is specifically bound to the thrombin. The aptamer-bindable substance 1 is the thrombin. The electrode reactive substance 2 is ferrocene. Further, the linker 14 is a branched type polyethylene glycol. The electrode 5 is a gold electrode and is connected to an electrochemical measurement device 13 together with a counter electrode 12. Prior to the detection of thrombin, which is a target substance, the probe 4 was immobilized to the electrode 5 via the linker 14. In this Example, a 10 mmol/L phosphate buffer (pH7.4) in which 0.1 mol/L sodium chloride was dissolved was provided as a measurement solution.

(i) Probe Provision Step

In this Example, first, the gold electrode 5 to which the probe 4 was immobilized on the surface thereof was immersed in a single-stranded DNA solution having a sequence of a thrombin aptamer, and the single-stranded DNA was immobilized to the thrombin serving as an aptamer-bindable substance 1.

(ii) Washing Step

In this Example, next, the gold electrode 5 to which the single-stranded DNA has already been immobilized was washed with PBS, and then the gold electrode 5 was immersed in the measurement solution.

(iii) Aptamer Binding State Detection Step

Next, the gold electrode 5 was connected to a working electrode terminal of the electrochemical measurement device 13, a platinum wire was connected to a counter electrode terminal, and the silver/silver chloride electrode was connected to a reference electrode terminal to measure the oxidation current of the ferrocene by differential pulse voltammetry.

(iv) Separation Step

Next, an aqueous solution of thrombin was added to the measurement solution so as to obtain the resultant having a final concentration of 500 nmol/L, and the resultant was incubated at room temperature for 2 hours.

(v) Detection Step

Next, the oxidation current of ferrocene was measured again by differential pulse voltammetry with the gold electrode as a working electrode.

The oxidation current of ferrocene of the (v) aptamer separation state detection step was increased about 50% as compared to that of the (iii) aptamer binding state detection step.

It is to be noted that, when the same steps as the steps (i) to (v) except that a 10 mmol/L phosphate buffer (pH7.4) in which 0.1 mol/L sodium perchlorate was dissolved was used as a measurement solution instead of a 10 mmol/L phosphate buffer (pH7.4) in which 0.1 mol/L sodium chloride was dissolved were performed, similar measurement results as described above were obtained. The same applies to Example 2.

### Example 2

In this Example, the oxidation current of ferrocene was measured using the same apparatus as Example 1 in the same manner as in Example 1 except that a bovine blood serum albumin aqueous solution (final concentration of 500 nmol/L) was used instead of a thrombin (target substance) aqueous solution in the (iv) separation step. In this Comparative Example, the oxidation current of ferrocene did not change between the (iii) aptamer binding state detection step and the (v) aptamer separation detection step. From the results of Examples 1 and 2, it can be confirmed that a target substance can be detected specifically by the method and apparatus of the present invention with an increase in the current value of ferrocene as an indicator.

### Example 3

In this Example, using the aptamer screening apparatus of the present invention illustrated in FIG. 6A, an aptamer that is specifically bound to thrombin was recovered by the screening method of the present invention. As shown in FIG. 6A, the aptamer screening apparatus of the present invention used in this Example includes a probe 4 in which an aptamer-bindable substance 1 that is specifically bound to an aptamer 6 and an electrode reactive substance 2 are each bound to an end of a linker 14; an electrode 5; and an electron transfer detecting unit 13. Specifically, the aptamer-bindable substance 1 is thrombin. The electrode reactive substance 2 is methylene blue. Further, the linker 14 is a branched type polyethylene glycol. The electrode 5 is a gold electrode (1 cm²) and is connected to the electrochemical measurement device 13 together with a counter electrode 12. Prior to the following (i) aptamer addition step, the probe 4 was immobilized to the electrode 5 via the linker 14. In this Example, as a sample solution containing an aptamer candidate substance, a sample solution obtained by dissolving a single-stranded DNA having a sequence of thrombin aptamer and a primer sequence and a single-stranded DNA having poly A with the same base number as the thrombin aptamer and a primer sequence into a binding buffer was prepared. The binding buffer is a 10 mmol/L phosphate buffer (pH7.4) containing 1 mol/L sodium chloride and 5 mM magnesium chloride. The concentration of each of the single-stranded DNAs in the sample solution was 100 mmol/L.

(i) First Step

100 μL of the sample solution was fractionated and dropped onto the surface of the gold electrode.

(ii) Washing Step

After the electrode was incubated at 37° C. for 1 hour, the surface of the electrode was washed with the binding buffer.

(iii) Second Step

Next, a binding buffer in which 1 μmol/L of thrombin was dissolved was dropped onto the electrode and the electrode was incubated at 37° C. for 5 hours.

(iv) Third Step

Then, a solution remained on the electrode was recovered with a pipet.

(v) Analysis Step

The nucleic acid in the solution recovered was amplified by PCR, and then the amplified DNA was analyzed with a DNA sequencer. From the analysis result, it was confirmed that only the single-stranded DNA having the thrombin aptamer sequence and the primer sequence was detected.

### INDUSTRIAL APPLICABILITY

Since the method of detecting a target substance of the present invention does not require an aptamer as an essential component in a probe for detecting a target substance, the design of the probe is simple. Therefore, for example, this brings advantages of making the production of the probe more efficient and reducing the production cost. The probe of the present invention makes it possible to achieve the detection method of the present invention. Further, the probe of the present invention makes it possible to provide the target substance detection apparatus used for the detection method of the present invention and the aptamer screening method and the aptamer screening apparatus with which the aptamer used for the detection method of the present invention can easily be obtained. Further, since the detection method of the present invention shows a signal-increasing type reaction in which the detection value of the electron transfer increases as the target substance increases, it brings advantages in that the procedure of the target substance detection is simple and a high S/N ratio is achieved. Further, the detection method of the present invention can be applied to various aptamers and target substances regardless of the conformational change of the aptamer, for example. Therefore, the detection method of the present invention achieves very high general versatility and has very high industrial use values.

The invention of the present application was described above with reference to the Embodiments and Examples. However, the invention of the present application is not limited to the above-described Embodiments and Examples. Various changes that can be understood by those skilled in the art can be made in the configurations and details of the invention within the scope of the invention.

This application claims priority from Japanese Patent Application No. 2009-065319 filed on Mar. 17, 2009. The entire subject matter of the Japanese Patent Applications is incorporated herein by reference.

### EXPLANATION OF REFERENCE NUMERALS

1 aptamer-bindable substance
2 labeling substance (electrode reactive substance)
3 target substance detection apparatus
4 probe
5 electrode
6 aptamer, aptamer candidate substance
7 single-stranded nucleic acid fragment
8 target substance
9 non-target substance
12 counter electrode
13 electron transfer detecting unit (electrochemical measurement device)
14 linker

1. A method of detecting a target substance, comprising:
a step of providing a probe comprising an aptamer-bindable substance, a labeling substance, and a linker immobilizable to a support wherein the aptamer-bindable substance and the labeling substance are each bound to the linker and an aptamer is specifically bound to the aptamer-bindable substance; and
a step of detecting the target substance by separating the aptamer from the aptamer-bindable substance through binding between a target substance in a sample and the aptamer and detecting separation of the aptamer based on the labeling substance wherein the probe is immobilized to a support via the linker.

2. The method according to claim 1, wherein the support is an electrode, and the separation of the aptamer in the step of detecting is detected as an electrical reaction between the labeling substance and the electrode.

3. The method according to claim 2, wherein the labeling substance is an electrode reactive substance, and the separation of the aptamer in the step of detecting is detected by comparing a detection value of an electron transfer between the electrode reactive substance and the electrode prior to supply of the target substance to the probe and a detection value of the electron transfer between the electrode reactive substance and the electrode after supply of the target substance to the probe.

4. The method according to claim 3, wherein a known detection value is used as the detection value of the electron transfer between the electrode reactive substance and the electrode prior to supply of the target substance to the probe, and the known detection value is compared with the detection value of the electron transfer between the electrode reactive substance and the electrode after supply of the target substance to the probe.

5. The method according to claim 3, wherein
the electrode reactive substance is at least one of a substance having an oxidation-reduction potential and a catalyst.

6. The method according to claim 5, wherein the electrode reactive substance is the catalyst, and the electron transfer between the catalyst and the electrode is performed through an electron transfer mediator.

7. The method according to claim 5, wherein the electrode reactive substance is the substance having an oxidation-reduction potential, and an oxidation-reduction potential of the substance having an oxidation-reduction potential is between −0.6 V to +1.4 V when the oxidation-reduction potential is a standard electrode potential according to a standard hydrogen electrode.

8. The method according to claim 1, wherein the aptamer-bindable substance has an epitope that is identical to or similar to the whole or a part of the target substance, and the aptamer specifically binds to the epitope.

9. The method according to claim 1, wherein the aptamer has a double-stranded nucleic acid part, and the aptamer-bindable substance has a double-stranded nucleic acid binding part that specifically binds to the double-stranded nucleic acid part.

10. The method according to claim 9, wherein
the double-stranded nucleic acid binding part is at least one of an intercalator and a nucleic acid binding protein.

11. The method according to claim 3, wherein the aptamer-bindable substance also serves as the electrode reactive substance.

12. The method according to claim 1, wherein the linker is at least one of a hydrophilic polymer and a hydrophilic oligomer.

13. The method according to claim 12, wherein at least one of the hydrophilic polymer and the hydrophilic oligomer has a negative charge.

14. The method according to claim 1, comprising a step of removing an aptamer that is not bound to the aptamer-bindable substance prior to the step of detecting.

15. A method of screening an aptamer comprising:
a first step of supplying an aptamer candidate substance to a probe;
a second step of separating the aptamer candidate substance from the probe by supplying a target substance to the probe immobilized to a support and binding the aptamer candidate substance that is bound to the probe to the target substance; and
a third step of recovering the aptamer candidate substance separated,
wherein said probe comprises an aptamer-bindable substance, a labeling substance, and a linker immobilizable to a support,
wherein the aptamer-bindable substance and the labeling substance are each bound to the linker, the aptamer-bindable substance and the labeling substance are immobilizable to the support via the linker, and an aptamer is specifically bindable to the aptamer-bindable substance.

16. The method according to claim 15, wherein the aptamer candidate substance is an aptamer candidate nucleic acid, the method further comprises:
a fourth step of amplifying the aptamer candidate nucleic acid recovered in the third step, and wherein the aptamer candidate nucleic acid amplified in the fourth step is provided as the aptamer candidate nucleic acid in the first step, and a procedure from the first step to the fourth step is performed repeatedly.

17. The method according to claim 16, wherein in addition to the aptamer candidate nucleic acid amplified, an aptamer candidate nucleic acid other than the aptamer candidate nucleic acid amplified is supplied as the aptamer candidate nucleic acid in the first step.

18. The method according to claim 15, further comprising:
a step of detecting separation of the aptamer candidate substance in the second step based on the labeling substance, wherein the support is an electrode, and the separation in the step of detecting is detected as an electrical reaction between the labeling substance and the electrode.

19. The method according to claim 18, wherein the probe comprising an electrode reactive substance as the labeling substance is used, and separation of the aptamer candidate substance in the second step is detected by comparing a detection value of an electron transfer between the electrode reactive substance and the electrode prior to supply of the target substance to the probe and a detection value of the electron transfer between the electrode reactive substance and the electrode after supply of the target substance to the probe.

20. The method according to claim 19, wherein a known detection value is used as the detection value of the electron transfer between the electrode reactive substance and the electrode prior to supply of the target substance to the probe, and the known detection value is compared with the detection value of the electron transfer between the electrode reactive substance and the electrode after supply of the target substance to the probe.

21. The method according to claim 19, wherein a screening status of an aptamer candidate nucleic acid is monitored by detecting the electron transfer between the electrode reactive substance and the electrode.

22. The method according to claim 21, wherein the aptamer candidate substance is an aptamer candidate nucleic acid, the method further comprises:
a fourth step of amplifying the aptamer candidate nucleic acid recovered in the third step, and wherein the aptamer candidate nucleic acid amplified in the fourth step is supplied as the aptamer candidate nucleic acid in the first step, a procedure from the first step to the fourth step is performed repeatedly, and a termination point in the case of repeating the procedure from the first step to the fourth step is determined by monitoring the screening status.

23. The method according to claim 19, wherein the electrode reactive substance is a catalyst, and the electron transfer between the catalyst and the electrode is performed through an electron transfer mediator.

24. The method according to claim 15, further comprising:
a fifth step of removing an aptamer candidate nucleic acid that is not bound to the probe prior to the second step.

25. The method according to claim 15, further comprising:
a sixth step of removing the aptamer candidate substance that is not bound to the target substance.

26. The method according to claim 1, wherein the aptamer is obtained by the method of screening an aptamer comprising:
a first step of supplying an aptamer candidate substance to a probe comprising:
an aptamer-bindable substance;
a labeling substance; and
a linker immobilizable to a support, wherein the aptamer-bindable substance and the labeling substance are each bound to the linker, the aptamer-bindable substance and the labeling substance are immobilizable to the support via the linker, and an aptamer is specifically bindable to the aptamer-bindable substance;
a second step of separating the aptamer candidate substance from the probe by supplying a target substance to the probe immobilized to a support and binding the aptamer candidate substance that is bound to the probe to the target substance; and
a third step of recovering the aptamer candidate substance separated.
